SEQUENCE LISTING (6 sequences)

SEQ ID NO: 1 - protein, 559 aa, Gloeophyllum trabeum
QSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGAPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 2 - protein, 556 aa, Gloeophyllum abietinum
QSVDSYVSSEGPIAKAGVLANIGPNGSKASGASAGVVVASPSTSDPDYWYTWTRDSSLVFKSLIDQYTTGIDGTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGIASNAAVATGRYPEDSYQGGNPWYLTTFAVAEQLYDALNVWESQGSLEVTSTSLAFFQQFSSGVTAGTYSSSSSTYSTLTSAIKSFADGFVAVNAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 3 - protein, 556 aa, Trametes cingulata
QSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

SEQ ID NO: 4 - protein, 555 aa, Pycnoporus sanguineus
QSSAVDAYVASESPIAKQGVLNNIGPNGSKAHGAKAGIVVASPSTENPDYLYTWTRDSSLVFKLLIDQFTSGDDTSLRGLIDDFTSAEAILQQVSNPSGTVSTGGLGEPKFNIDETAFTGAWGRPQRDGPALRATSIIRYANWLLDNGNTTYVSNTLWPVIQLDLDYVADNWNQSTFDLWEEVDSSSFFTTAVQHRALREGATFASRIGQSSVVSGYTTQADNLLCFLQSYWNPSGGYVTANTGGGRSGKDSNTVLTSIHTFDPAAGCDAATFQPCSDKALSNLKVYVDAFRSIYTINNGIASNAAVATGRYPEDSYMGGNPWYLTTSAVAEQLYDALYVWDQLGGLNVTSTSLAFFQQFASGLSTGTYSASSSTYATLTSAIRSFADGFLAINAKYTPADGGLAEQYSRNDGTPLSAVDLTWSYAAALTAFAAREGKTYGSWGAAGLTVPASCSGGGGATVAVTFNVQATTVFGENIYITGSVAALQNWSPDNALILSAANYPTWSITVNLPANTVVQYKYIRKFNGQVTWESDPNNQITTPSGGSFTQNDVWR

SEQ ID NO: 5 - protein, 583 aa, Artificial
  Hybrid alpha-amylase
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 6 - protein, 587 aa, Aspergillus terreus
LTPAEWRSQSIYFLLTDRFGRTDNSTTAACDTSDRVYCGGSWQGIINQLDYIQGMGFTAIWITPVTGQFYENTGDGTSYHGYWQQDIYDLNYNYGTAQDLKNLANALHERGMYLMVDVVANHMGYDGAGNTVDYSVFNPFSSSSYFHPYCLISNYDNQTNVEDCWLGDTTVSLPDLDTTSTAVRNIWYDWVADLVANYSIDGLRVDTVKHVEKDFWPGYNSAAGVYCVGEVYSGDPAYTCPYQNYMDGVLNYPIYYQLLYAFESSSGSISDLYNMISSVASSCKDPTLLGNFIENHDNPRFASYTSDYSQAKNVITFIFLSDGIPIVYAGQEQHYSGGSDPANREATWLSGYSTSATLYTWIATTNQIRSLAISKDAGYVQAKNNPFYSDSNTIAMRKGTTAGAQVITVLSNKGASGSSYTLSLSGTGYSAGATLVETYTCTTVTVDSSGNLPVPMTSGLPRVFVPSSWVNGSALCNTECTAATSISVLFEELVTTTYGENIYLSGSISQLGSWNTASAVALSASQYTSSNPEWYVSVTLPVGTSFQYKFIKKGSDGSVVWESDPNRSYTVPAGCEGATVTVADTWR

(19) United States
(12) Patent Application Publication
(10) Pub. No.: US 2020/0248208 A1
(43) Pub. Date: Aug. 6, 2020

(54) Yeast Strains Suitable For Saccharification And Fermentation Expressing Glucoamylase And/Or Alpha-Amylase

(71) Applicant: Novozymes A/S, Bagsvaerd (DK)

(72) Inventors: Michael G. Catlett, Davis, CA (US); Jennifer Headman, Franklinton, NC (US); Shiro Fukuyama, Chiba (JP)

(73) Assignee: Novozymes A/S, Bagsvaerd (DK)

(21) Appl. No.: 15/774,519

(22) PCT Filed: Nov. 14, 2016
(86) PCT No.: PCT/US2016/061887

§ 371 Date: May 8, 2018

(51) Int. Cl. C12P 7/10; C12N 9/34; C12N 9/30

(52) U.S. Cl. CPC C12P 7/10; C12N 9/2428; C12R 1/865; C12Y 302/01001; C12Y 302/01003; C12N 9/242

(57) ABSTRACT

The present invention provides in a process of producing ethanol from starch-containing material comprising: (a) saccharifying the starch-containing material; and (b) fermenting using a fermentation organism; wherein saccharification and/or fermentation is done in the presence of at least a glucoamyl ase and optionally an alpha-amylase; the fermenting organism is Saacaromyces cerevisiae; and wherein a glucoamylase and/or an alpha-amylase is expressed from the fermenting organism. Further the invention provides yeast strains specifically developed for the process of the invention.

### REFERENCE TO SEQUENCE LISTING

This application contains a Sequence Listing in computer readable form. The computer readable form is incorporated herein by reference.

### FIELD OF THE INVENTION

The present invention relates to processes for producing ethanol from starch containing material and yeast strain developed for such processes.

### BACKGROUND OF THE INVENTION

Processes of producing ethanol from starch-containing material are well-known in the art and used commercially today. The production of ethanol as a bio-fuel has become a major industry, with in excess of 21 billion gallons of ethanol being produced worldwide in 2012.

When producing ethanol, starch is conventionally converted into dextrins using a liquefying enzyme (e.g., Bacillus alpha-amylase) at temperatures above the initial gelatinization temperature of starch. The generated dextrins are hydrolyzed into sugars using a saccharifying enzyme (e.g., glucoamylase) and fermented into the desired fermentation product using a fermenting organism such as a yeast strain derived from Saacaromyces cerevisiae. Typically hydrolysis and fermentation are done in a simultaneous saccharification and fermentation (SSF) step.

Another type of process is also used commercially today. Starch is converted into sugars by enzymes at temperatures below the initial gelatinization temperature of the starch in question and converted into ethanol by yeast, typically derived from Saacaromyces cerevisiae. This type of process is referred to as a raw starch hydrolysis (RSH) process, or alternatively a “one-step process” or “no cook” process.

Yeast which are used for production of ethanol for use as fuel, such as in the corn ethanol industry, require several characteristics to ensure cost effective production of the ethanol.

These characteristics include ethanol tolerance, low by-product yield, rapid fermentation, and the ability to limit the amount of residual sugars remaining in the ferment. Such characteristics have a marked effect on the viability of the industrial process.

Yeast of the genus Saacaromyces exhibit many of the characteristics required for production of ethanol. In particular, strains of Saacaromyces cerevisiae are widely used for the production of ethanol in the fuel ethanol industry. Strains of Saacaromyces cerevisiae that are widely used in the fuel ethanol industry have the ability to produce high yields of ethanol under fermentation conditions found in, for example, the fermentation of corn mash. An example of such a strain is the yeast used in commercially available ethanol yeast product called Ethanol Red™.

Strains of Saacaromyces cerevisiae are used in the fuel ethanol industry to ferment sugars such as glucose, fructose, sucrose and maltose to produce ethanol via the glycolytic pathway. These sugars are obtained from sources such as corn and other grains, sugar juice, molasses, grape juice, fruit juices, and starchy root vegetables and may include the breakdown of cellulosic material into glucose.

Although strains of Saacaromyces cerevisiae currently used in the fuel ethanol industry are well suited to ethanol production, there is an increasing need for improvements in the efficiency of ethanol production owing to the increased demand for ethanol as a fuel, and the increased availability of starch in new strains of corn.

There is therefore a need for new strains of Saacaromyces capable of improving the efficiency of ethanol production in industrial scale fermentation.

Further, despite significant improvement of ethanol production processes over the past decade there is still a desire and need for providing further improved processes of producing ethanol from starch-containing material that, e.g., can provide a higher ethanol yield.

### SUMMARY OF THE INVENTION

The invention provides in a first aspect a process of producing ethanol from starchcontaining material comprising:

(a) saccharifying the starch-containing material; and

(b) fermenting using a fermentation organism;

wherein
saccharification and/or fermentation is done in the presence of at least a glucoamylase and optionally an alpha-amylase;
the fermenting organism is Saacaromyces cerevisiae;
   and wherein a glucoamylase and/or an alpha-amylase is expressed from the fermenting organism.

The invention also provides in a second aspect yeast strains comprising one or more expression constructs encoding a glucoamylase and/or an alpha-amylase, wherein the yeast is derived from a parent strain selected from MBG4851, MBG4931, MBG4911, MBG4913 and MBG4914, and wherein the glucoamylase is selected from glucoamylases obtainable from Gloeophyllum, Pycnoporous, or Trametes.

In a third aspect the invention provides a yeast strain comprising one or more expression constructs encoding a glucoamylase and/or an alpha-amylase, wherein the yeast is derived from a parent strain selected from MBG4851, MBG4931, MBG4911, MBG4913 and MBG4914, and wherein the alpha-amylase is selected from a Rhizomucor pusillus or Aspergillus terreus alpha-amylase.

### BRIEF DESCRIPTION OF DRAWINGS

FIG. 1 shows performance of yeast expressing alpha-amylase in a raw starch ethanol process at 55 hours in bottle scale.

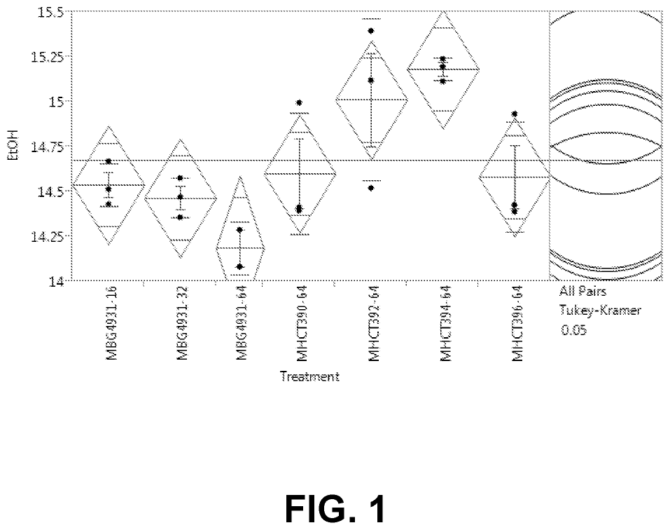

FIG. 2 shows performance of yeast expressing alpha-amylase in a raw starch ethanol process at 73 hours in bottle scale.

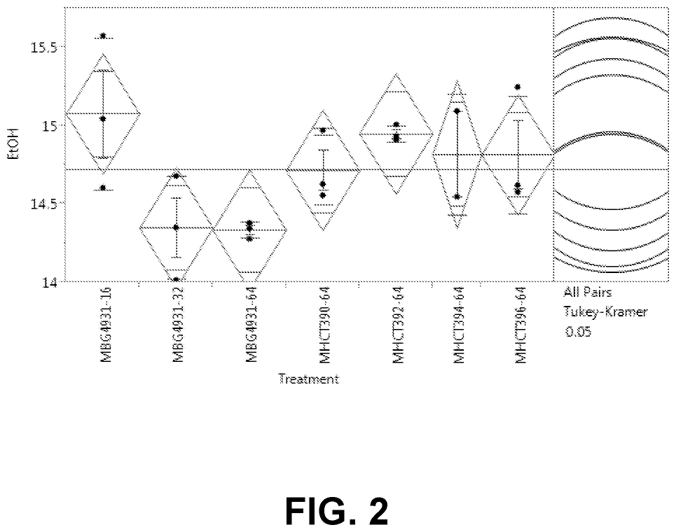

FIG. 3 shows performance of yeast expressing alpha-amylase in a raw starch ethanol process at 72 hours in tube scale.

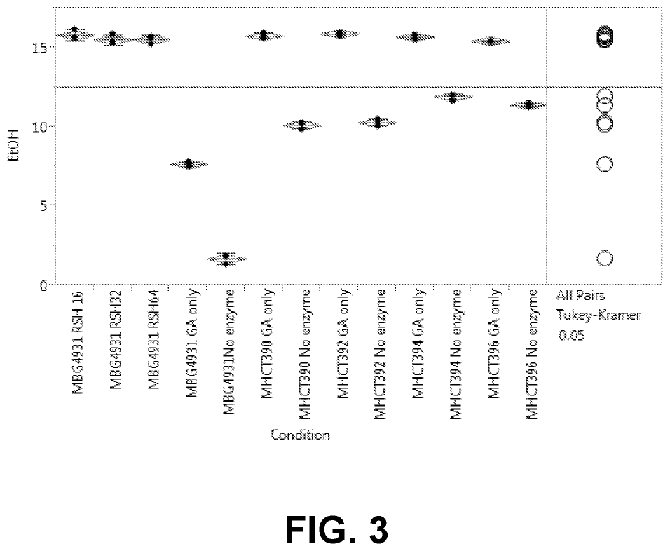

FIG. 4 shows performance (EtOH yield and glucose) at 44.4 hours of glucoamylase expressing yeast in SSF.

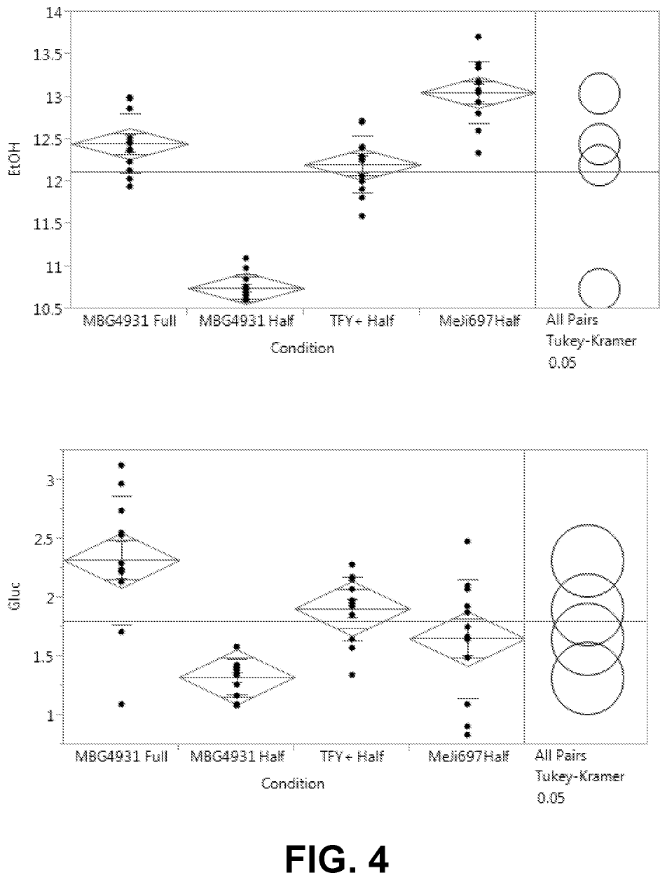

FIG. 5 shows performance (EtOH yield and glucose) at 60 hours of glucoamylase expressing yeast in SSF.

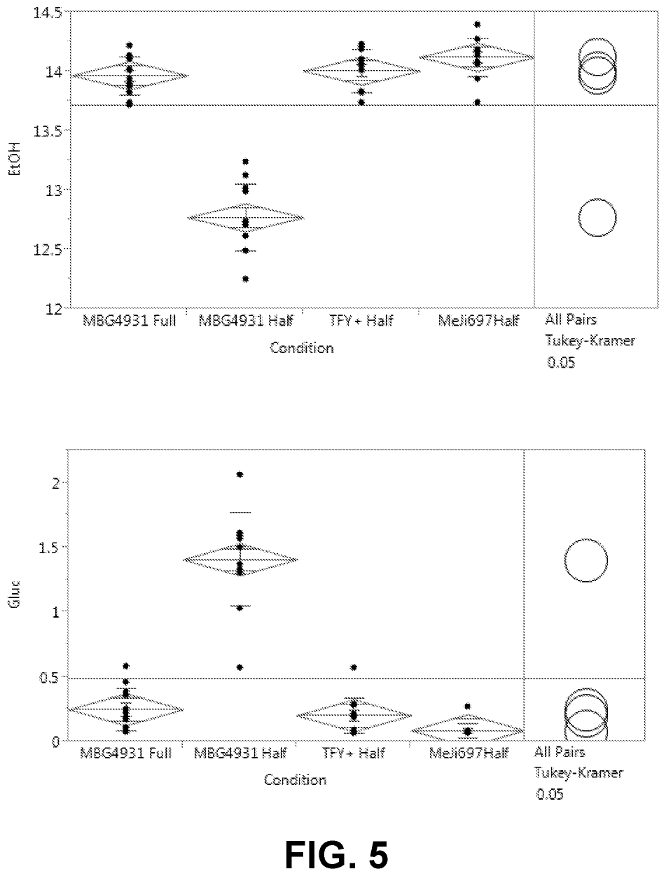

FIG. 6 shows a generalized diagram of the expression cassette at the XII-5 integration site. The dominant selection marker was either kanamycin or nourseothricin resistance. The “Gene of Interest” is, for example, an alpha-amylase or glucoamylase.

FIG. 7 shows performance of glucoamylase expressing yeast in SSF compared to control ER and parent background strains at 54 hours.

### DEFINITIONS

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g., several) amino acids absent from the amino and/or carboxyl terminus of a mature polypeptide or domain; wherein the fragment has pullulanas activity.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g. a host cell may be genetically modified to express the polypeptide of the invention. The fermentation broth from that host cell will comprise the isolated polypeptide.

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having protease activity.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

### DETAILED DESCRIPTION OF THE INVENTION

### Yeast of the Invention

The majority of the world's fuel ethanol is produced by industrial scale fermentation of starch-based sugars, in substrates such as corn mash. During industrial scale fermentation, the yeast encounter various physiological challenges including variable concentrations of sugars, high concentrations of yeast metabolites such as ethanol, glycerol, organic acids, osmotic stress, as well as potential competition from contaminating microbes such as wild yeasts and bacteria. As a consequence, many Saacaromyces strains are not suitable for use in industrial fermentation. The most widely used commercially available industrial strain of Saccharomyces (i.e. for industrial scale fermentation) is the Saacaromyces cerevisiae strain used, for example, in the product ETHANOL RED™. This strain is well suited to industrial ethanol production; however improved strains of Saacaromyces cerevisiae are needed.

Improved Saacaromyces cerevisiae yeast strains have been developed based on the following parent strains developed by breeding:

Strain no. V14/004037 (i.e., Saacaromyces cerevisiae MBG4851 and described in WO2015/143317 and WO2015/143324, each incorporated herein by reference);

Strain no. V15/004036 (i.e., Saacaromyces cerevisiae MBG4931 disclosed in WO2016/153924, incorporated herein by reference); and

Strains no. V15/001459, V15/001460, V15/001461 (i.e., Saacaromyces cerevisiae MBG4911, MBG4913, and MBG4914 disclosed in WO2016/138437, incorporated herein by reference).

The above strains have all previously been deposited under the Budapest treaty. Strain V14/004037 was deposited on 17 Feb. 2014 at the National Measurement Institute, 1/153 Bertie Street, Port Melbourne, Victoria 3207, Australia under the Budapest Treaty and was designated accession number V14/004037. Strain V15/004036 was deposited on 19 February 2015 at the National Measurement Institute, 1/153 Bertie Street, Port Melbourne, Victoria 3207, Australia under the Budapest Treaty and was designated accession number V15/004036. V15/001459, V15/001460, V15/001461 (i.e., Saacaromyces cerevisiae MBG4911, MBG4913, and MBG4914) were deposited by Microbiogen Pty Ltd, Unit E2, Lane Cove Business Park, 16 Mars Road, Lane Cove, NSW 2066, Australia under the terms of the Budapest Treaty with the National Measurement Institute, Victoria, Australia) and given the following accession number:

| Deposit | Accession Number | Date of Deposit |
|---|---|---|
| MBG4911 | V15/001459 | Jan. 13, 2015 |
| MBG4913 | V15/001460 | Jan. 13, 2015 |
| MBG4914 | V15/001461 | Jan. 13, 2015 |

The yeast strains according to the invention have been generated in order to improve ethanol yield and to improve process economy by cutting enzyme costs since part or all of the necessary enzymes needed to hydrolyse starch will be produced by the yeast organism.

One aspect of the present invention therefore relates to yeast strains comprising one or more expression constructs encoding a glucoamylase and/or an alpha-amylase, wherein the yeast is derived from a parent strain selected from MBG4851, MBG4931, MBG4911, MBG4913 and MBG4914; and wherein the glucoamylase is selected from glucoamylases obtainable from Gloeophyllum, Pycnoporous, Trametes.

Another aspect of the present invention therefore relates to yeast strain comprising one or more expression constructs encoding a glucoamylase and/or an alpha-amylase, wherein the yeast is derived from a parent strain selected from MBG4851, MBG4931, MBG4911, MBG4913and MBG4914; and wherein the alpha-amylase is selected from a Rhizomucor pusillus or Aspergillus terreus alpha-amylase.

In one embodiment the the glucoamylase is selected from a Gloeophyllum trabeum, Gloeophyllum sepiarium, or Gloeophyllum abietinum glucoamylase.

In another embodiment the glucoamylase is selected from the group consisting of:

(i) a glucoamylase comprising the polypeptide of SEQ ID NO: 1 or SEQ ID NO: 2;
(ii) a glucoamylase comprising an amino acid sequence having at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 1 or SEQ ID NO: 2.

In one embodiment the the glucoamylase is the Gloeophyllum trabeum glucoamylase shown in SEQ ID NO: 1 having one of the following substitutions: V59A; S95P; A121P; T119W; S95P+A121P; V59A+S95P; S95P+T119W; V59A+S95P+A121P; or S95P+T119W+A121P, especially S95P+A121P; and wherein the glucoamylase has at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 1.

In one particular embodiment the glucoamylase is selected from a Trametes cingulata glucoamylase. More particularly the glucoamylase is selected from the group consisting of:

(i) a glucoamylase comprising the polypeptide of SEQ ID NO: 3;
(ii) a glucoamylase comprising an amino acid sequence having at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 3.

In one particular embodiment the glucoamylase is selected from a Pycnoporus sanguineus glucoamylase. More particularly the glucoamylase is selected from the group consisting of:

(i) a glucoamylase comprising the polypeptide of SEQ ID NO: 4;
(ii) a glucoamylase comprising an amino acid sequence having at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 4.

In another particular embodiment the alpha-amylase is Rhizomucor pusillus alpha-amylase with an Aspergillus niger glucoamylase linker and starch-binding domain (SBD) as shown in SEQ ID NO: 5, preferably one having at least one of the following substitutions or combinations of substitutions: D165M; Y141W; Y141R; K136F; K192R; P224A; P224R; S123H+Y141W; G20S+Y141W; A76G 30 Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C, especially G128D+D143N (using SEQ ID NO: 5 for numbering), and wherein the alpha-amylase has at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 5.

In another particular embodiment the alpha-amylase is Aspergillus terreus alpha-amylase selected from the group consisting of:

(i) an alpha-amylase comprising the polypeptide of SEQ ID NO: 6;
(ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 6.

In one particular embodiment the yeast strain is derived from the parent strain MBG4911. In another particular embodiment the yeast strain is derived from the parent strain MBG4851. In another particular embodiment the yeast strain is derived from the parent strain MBG4931.

In an embodiment the parent yeast strain expressing glucoamylase is MBG4911. In an embodiment the parent yeast strain expresses Pycnoporus glucoamylase, particularly a Pycnoporus sanguineus glucoamylase. In a specific embodiment the MBG4911 strain expresses the Pycnoporus sanguineus glucoamylase shown in SEQ ID NO: 4. This latter mentioned strain is referred to as AgJg013. Contemplated are also strains having properties that are about the same as that of AgJg013.

In an embodiment the parent yeast strain expressing alpha-amylase is MBG4911. In an embodiment the parent yeast strain expresses Rhizomucor alpha-amylase. In a preferred embodiment the MBG4911 strain expresses a Rhizomucor pusillus alpha-amylase, in particular a Rhizomucor pusillus alpha-amylase with an Aspergillus niger glucoamylase linker and starch-binding domain (SBD), such as the one shown in SEQ ID NO: 5. In a specific embodiment the MBG4911 strain expresses the alpha-amylase shown in SEQ ID NO: 5 comprising the following substitutions: G128D+D143N (i.e., PE096 alpha-amylase). One example of the latter mentioned strain is referred to as MLBA795. Contemplated are also strains having properties that are about the same as that of MLBA795.

In an embodiment the parent yeast strain expressing glucoamylase and alpha-amylase is MBG4911. In an embodiment the parent yeast strain expresses a Pycnoporus glucoamylase, particularly a Pycnoporus sanguineus glucoamylase (e.g., SEQ ID NO: 4) and a Rhizomucor pusillus alpha-amylase, in particular a Rhizomucor pusillus alpha-amylase with an Aspergillus niger glucoamylase linker and starch-binding domain (SBD) shown in SEQ ID NO: 5. In a specific embodiment the parent yeast strain expresses the glucoamylase shown in SEQ ID NO: 4 and alpha-amylase shown in SEQ ID NO: 5 comprising the following substitutions: G128D+D143N (i.e., PE096 alpha-amylase). Examples of the mentioned strain include MLBA821 and MLBA855. Contemplated are also strains having properties that are about the same as that of MLBA821 or MLBA855.

In an embodiment the parent yeast strain, e.g., MBG4911, expresses a glucoamylase and/or an alpha-amylases having at least 80%, at least 90%, at least 95%, at least 97%, at least 99% sequence identity to SEQ ID NO: 4 and/or SEQ ID NO: 5, respectively.

In an embodiment the parent yeast strain expressing glucoamylase is MBG4851. In an embodiment the parent yeast strain expresses a Gloeophyllum glucoamylase, particularly a Gloeophyllum trabeum or Gloeophyllum sepiarium (formally Gloeophyllum abietinum) glucoamylase. In a specific embodiment the MBG4851 strain expresses the Gloeophyllum trabeum glucoamylase shown in SEQ ID NO: 1. In another specific embodiment the MBG4851 strain expresses the Gloeophyllum sepiarium (formally Gloeophyllum abietinum) glucoamylase shown in SEQ ID NO: 2.

In an embodiment the parent yeast strain expressing alpha-amylase is MBG4851. In an embodiment the parent yeast strain expresses Rhizomucor alpha-amylase. In a preferred embodiment the MBG4851 strain expresses a Rhizomucor pusillus alpha-amylase, in particular a Rhizomucor pusillus alpha-amylase with an Aspergillus niger glucoamylase linker and starch-binding domain (SBD), such as the one shown in SEQ ID NO: 5. In a specific embodiment the MBG4851 strain expresses the alpha-amylase shown in SEQ ID NO: 5 comprising the following substitutions: G128D+D143N (i.e., PE096 alpha-amylase).

In an embodiment the parent yeast strain expressing glucoamylase and alpha-amylase is MBG4851. In an embodiment the parent yeast strain expresses a Gloeophyllum glucoamylase, particularly a Gloeophyllum trabeum or Gloeophyllum sepiarium (formally Gloeophyllum abietinum) glucoamylase (e.g., SEQ ID NO: 1 or 2, respectively) and a Rhizomucor pusillus alpha-amylase, in particular a Rhizomucor pusillus alpha-amylase with an Aspergillus niger glucoamylase linker and starch-binding domain (SBD) shown in SEQ ID NO: 5. In a specific embodiment the parent yeast strain expresses the glucoamylase shown in SEQ ID NO: 1 or 2, and alpha-amylase shown in SEQ ID NO: 5 comprising the following substitutions: G128D+D143N (i.e., PE096 alpha-amylase).

In an embodiment the parent yeast strain, e.g., MBG4851, expresses a glucoamylase having at least 80%, at least 90%, at least 95%, at least 97%, at least 99% sequence identity to SEQ ID NO: 1 or 2, and/or an alpha-amylases having at least 80%, at least 90%, at least 95%, at least 97%, at least 99% sequence identity to SEQ ID NO: 5.

In an embodiment the parent yeast strain expressing glucoamylase is MBG4931. In an embodiment the parent yeast strain expresses a Gloeophyllum glucoamylase, particularly a Gloeophyllum trabeum or Gloeophyllum sepiarium (formally Gloeophyllum abietinum) glucoamylase. In a specific embodiment the MBG4931 strain expresses the Gloeophyllum trabeum glucoamylase shown in SEQ ID NO: 1. In another specific embodiment the MBG4931 strain expresses the Gloeophyllum sepiarium (formally Gloeophyllum abietinum) glucoamylase shown in SEQ ID NO: 2. Examples of this latter mentioned strain include MEJ1697 and MEJ1705. Contemplated are also strains having properties that are about the same as that of MEJ1697 or MEJ1705.

In an embodiment the parent yeast strain expressing alpha-amylase is MBG4931. In an embodiment the parent yeast strain expresses Rhizomucor alpha-amylase. In a preferred embodiment the MBG4931 strain expresses a Rhizomucor pusillus alpha-amylase, in particular a Rhizomucor pusillus alpha-amylase with an Aspergillus niger glucoamylase linker and starch-binding domain (SBD), such as the one shown in SEQ ID NO: 5. In a specific embodiment the MBG4911 strain expresses the alpha-amylase shown in SEQ ID NO: 5 comprising the following substitutions: G128D+D143N (i.e., PE096 alpha-amylase). Examples of this latter mentioned strain include yMHCT394 and yMHCT396. Contemplated are also strains having properties that are about the same as that of yMHCT394 or yMHCT396.

In an embodiment the parent yeast strain expressing glucoamylase and alpha-amylase is MBG4931. In an embodiment the parent yeast strain expresses a Gloeophyllum glucoamylase, particularly a Gloeophyllum trabeum or Gloeophyllum sepiarium (formally Gloeophyllum abietinum) glucoamylase (e.g., SEQ ID NO: 1 or 2, respectively) and a Rhizomucor pusillus alpha-amylase, in particular a Rhizomucor pusillus alpha-amylase with an Aspergillus niger glucoamylase linker and starch-binding domain (SBD) shown in SEQ ID NO: 5. In a specific embodiment the parent yeast strain expresses the glucoamylase shown in SEQ ID NO: 1 or 2, and alpha-amylase shown in SEQ ID NO: 5 comprising the following substitutions: G128D+D143N (i.e., PE096 alpha-amylase).

In an embodiment the parent yeast strain, e.g., MBG4931, expresses a glucoamylase having at least 80%, at least 90%, at least 95%, at least 97%, at least 99% sequence identity to SEQ ID NO: 1 or 2, and/or an alpha-amylases having at least 80%, at least 90%, at least 95%, at least 97%, at least 99% sequence identity to SEQ ID NO: 5.

Production of Fermentation Products from Starch-Containing Materials

In one aspect the present invention relates to a process for producing a fermentation product, especially ethanol, from starch-containing material, which process includes a liquefaction step and sequentially or simultaneously performed saccharification and fermentation steps.

The invention relates to processes for producing fermentation products from starch-containing material comprising the steps of:

i) liquefying starch-containing material using an alpha-amylase:
ii) saccharifying the starch-containing material; and;
iii) fermenting using a fermentation organism;

wherein

saccharification and/or fermentation is done in the presence of at least a glucoamylase and optionally an alpha-amylase;

the fermenting organism is Saacaromyces cerevisiae ;

and wherein a glucoamylase and/or an alpha-amylase is expressed from the fermenting organism.

In one embodiment the liquefaction step is performed in the presence of at least a bacterial alpha-amylase, such as an alpha-amylase from Bacillus sp., particularly Bacillus stearothermophilus.

The fermentation product, such as especially ethanol, may optionally be recovered after fermentation, e.g., by distillation. Suitable starch-containing starting materials are listed in the section “Starch-Containing Materials”-section below. In an embodiment the starch-containing materials is corn or what. Contemplated enzymes are listed in the “Enzymes”-section below. The liquefaction is carried out in the presence of an alpha-amylase, preferably a bacterial alpha-amylase, especially Bacillus alpha-amylase, such as a Bacillus stearothermophilus alpha-amylase. The fermenting organism is preferably yeast, preferably a strain of Saccharomyces, especially a strain of Saacaromyces cerevisae. Suitable fermenting organisms are listed in the “Fermenting Organisms”-section above. In a preferred embodiment steps ii) and iii) are carried out sequentially or simultaneously (i.e., as SSF process).

In a particular embodiment, the process of the invention further comprises, prior to liquefaction step i), the steps of:

x) reducing the particle size of the starch-containing material, preferably by milling;

y) forming a slurry comprising the starch-containing material and water. The aqueous slurry may contain from 10-55 wt.-% dry solids, preferably 25-45 wt.-% dry solids, more preferably 30-40 wt.-% dry solids of starch-containing material. The slurry is heated to above the initial gelatinization temperature. Alpha-amylase, preferably bacterial alpha-amylase, may be added to the slurry. In an embodiment the slurry is also jet-cooked to further gelatinize the slurry before being subjected to an alpha-amylase in liquefaction step i).

The temperature during step (i) is above the initial gelatinization temperature, such as between 80-90° C., such as around 85° C.

In an embodiment liquefaction is carried out as a three-step hot slurry process. The slurry is heated to between 60-95° C., preferably between 80-90° C., and alpha-amylase is added to initiate liquefaction (thinning). Then the slurry is jet-cooked at a temperature between 95-140° C., preferably 105-125° C., for 1-15 minutes, preferably for 3-10 minutes, especially around 5 minutes. The slurry is cooled to 60-95° C., preferably 80-90° C., and more alpha-amylase is added to finalize hydrolysis (secondary liquefaction). The liquefaction process is usually carried out at pH 4.5-6.5, in particular at a pH between 5 and 6. Milled and liquefied starch is known as “mash”.

The saccharification in step ii) may be carried out using conditions well known in the art. For instance, a full saccharification process may last up to from about 24 to about 72 hours. In an embodiment a pre-saccharification step is done at 40-90 minutes at a temperature between 30-65° C., typically at about 60° C., followed by complete saccharification during fermentation in a simultaneous saccharification and fermentation step (SSF). Saccharification is typically carried out at temperatures from 30-70° C., such as 55-65° C., typically around 60° C., and at a pH between 4 and 5, normally at about pH 4.5.

The most widely used process in fermentation product production, especially ethanol production, is simultaneous saccharification and fermentation (SSF) process, in which there is no holding stage for the saccharification.

SSF may typically be carried out at a temperature between 25° C. and 40° C., such as between 28° C. and 36° C., such as between 30° C. and 34° C., such as around 32° C., when the fermentation organism is yeast, such as a strain of Saacaromyces cerevisiae , and the desired fermentation product is ethanol. In an embodiment fermentation is ongoing for 6 to 120 hours, in particular 24 to 96 hours.

Other fermentation products may be fermented at conditions and temperatures, well known to the skilled person in the art, suitable for the fermenting organism in question. According to the invention the temperature may be adjusted up or down during fermentation.

In another aspect the invention relates to processes for producing a fermentation product from starch-containing material without gelatinization of the starch-containing material (i.e., uncooked starch-containing material). According to the invention the desired fermentation product, such as ethanol, can be produced without liquefying the aqueous slurry containing the starch-containing material. In one embodiment a process of the invention includes saccharifying (milled) starch-containing material, especially granular starch, below the initial gelatinization temperature, preferably in the presence of a carbohydrate-source generating enzyme, preferably a glucoamylase, to produce sugars that can be fermented into the desired fermentation product by a suitable fermenting organism.

In this embodiment the desired fermentation product, especially ethanol, is produced from un-gelatinized (i.e., uncooked) milled starch-containing material, especially granular starch.

Accordingly, in this aspect the invention relates to processes of producing a fermentation product from starch-containing material, comprising the steps of:

(a) saccharifying starch-containing material at a temperature below the initial gelatinization temperature of said starch-containing material in the presence of at least a glucoamylase and optionally an alpha-amylase;

(b) fermenting using a fermenting organism;

wherein

saccharification and/or fermentation is done in the presence of at least a glucoamylase and optionally an alpha-amylase;

the fermenting organism is Saacaromyces cerevisiae;

and wherein a glucoamylase and/or an alpha-amylase is expressed from the fermenting organism.

In a preferred embodiment steps (a) and (b) are carried out simultaneously (i.e., one step fermentation) or sequentially. The fermentation product, such as especially ethanol, may optionally be recovered after fermentation, e.g., by distillation. Suitable starch-containing starting materials are listed in the section “Starch-Containing Materials”-section below. In a preferred embodiment the starch-containing material is granular starch. Contemplated enzymes are listed in the “Enzymes”-section below. A glucoamylase and/or an alpha-amylase may be present. Alpha-amylases used are preferably acidic alpha-amylases, preferably acid fungal alpha-amylases.

The term “below the initial gelatinization temperature” means below the lowest temperature where gelatinization of the starch commences. Starch heated in water typically begins to gelatinize between 50° C. and 75° C.; the exact temperature of gelatinization depends on the specific starch, and can readily be determined by the skilled artisan. Thus, the initial gelatinization temperature may vary according to the plant species, to the particular variety of the plant species as well as with the growth conditions. In the context of this invention the initial gelatinization temperature of a given starch-containing material is the temperature at which birefringence is lost in 5% of the starch granules using the method described by Gorinstein and Lii, 1992, Starch/Starke 44 (12): 461-466.

Before step (a) a slurry of starch-containing material, such as granular starch, having 10-55 wt.-% dry solids, preferably 25-45 wt.-% dry solids, more preferably 30-40 wt.-% dry solids of starch-containing material may be prepared. The slurry may include water and/or process waters, such as stillage (backset), scrubber water, evaporator condensate or distillate, side stripper water from distillation, or other fermentation product plant process water. Because the process of the invention is carried out below the initial gelatinization temperature and thus no significant viscosity increase takes place, high levels of stillage may be used if desired. In an embodiment the aqueous slurry contains from about 1 to about 70 vol.-% stillage, preferably 15-60% vol.-% stillage, especially from about 30 to 50 vol.-% stillage.

The starch-containing material may be prepared by reducing the particle size, preferably by dry or wet milling, to 0.05 to 3.0 mm, preferably 0.1-0.5 mm. After being subjected to a process of the invention at least 85%, at least 86%, at least 87%, at least 88%, at least 89%, at least 90%, at least 91%, at least 92%, at least 93%, at least 94%, at least 95%, at least 96%, at least 97%, at least 98%, or preferably at least 99% of the dry solids of the starch-containing material is converted into a soluble starch hydrolyzate.

The process of the invention is conducted at a temperature below the initial gelatinization temperature. Preferably the temperature at which step (a) is carried out is between 30-75° C., preferably between 45-60° C.

In a preferred embodiment step (a) and step (b) are carried out as a simultaneous saccharification and fermentation process (SSF). In such preferred embodiment the process is typically carried at a temperature between 25° C. and 40° C., such as between 28° C. and 36° C., such as between 30° C. and 34° C., such as around 32° C. According to the invention the temperature may be adjusted up or down during fermentation.

In an embodiment simultaneous saccharification and fermentation is carried out so that the sugar level, such as glucose level, is kept at a low level such as below 6 wt.-%, preferably below about 3 wt.-%, preferably below about 2 wt.-%, more preferred below about 1 wt.-%., even more preferred below about 0.5%, or even more preferred 0.25% wt.-%, such as below about 0.1 wt.-%. Such low levels of sugar can be accomplished by simply employing adjusted quantities of enzyme and fermenting organism. A skilled person in the art can easily determine which quantities of enzyme and fermenting organism to use. The employed quantities of enzyme and fermenting organism may also be selected to maintain low concentrations of maltose in the fermentation broth. For instance, the maltose level may be kept below about 0.5 wt.-% or below about 0.2 wt.-%.

The process of the invention may be carried out at a pH in the range between 3 and 7, preferably from pH 3.5 to 6, or more preferably from pH 4 to 5.

According to the invention specifically developed yeast strains are used as fermenting organisms wherein a glucoamylase and/or an alpha-amylase is expressed from the fermenting organism. Suitable specific strains are developed from preferred parent strains selected from:

MBG4931 (deposited under Accession No. V15/004036 at National Measurement Institute, Victoria, Australia) or a fermenting organism having properties that are about the same as that of Saacaromyces cerevisiae MBG4931 or a derivative of Saacaromyces strain V15/004036 having defining characteristics of strain V15/004036;

MBG4851 (deposited under Accession No. V14/004037 at National Measurement Institute, Victoria, Australia) or a fermenting organism strain having properties that are about the same as that of Saacaromyces cerevisiae MBG4851, or a derivative of Saacaromyces strain V14/004037 having the defining characteristics of strain V14/004037;

MBG4911 (deposited as V15/001459 at National Measurement Institute, Victoria, Australia) or a fermenting organism strain having properties that are about the same as that of Saccharomyces cerevisiae MBG4911;

MBG4913 (deposited as V15/001460 at National Measurement Institute, Victoria, Australia) or a fermenting organism strain having properties that are about the same as that of Saccharomyces cerevisiae is MBG4913; and

MBG4914 (deposited as V15/001461 at National Measurement Institute, Victoria, Australia) or a fermenting organism strain having properties that are about the same as that of Saacaromyces cerevisiae is MBG4914.

According to a preferred embodiment of the processes of the invention the yeast fermenting organism expresses a glucoamylase, particularly the glucoamylase expressed from the fermenting organism is a Gloeophyllum glucoamylase, preferably Gloeophyllum trabeum, Gloeophyllum sepiarium, Gloeophyllum abietinum glucoamylase, a Trametes glucoamylase, preferably a Trametes cingulata glucoamylase, a Pycnoporus glucoamylase, particularly a Pycnoporus sanguineus glucoamylase.

In one embodiment of the process of the invention the glucoamylase is selected from the group consisting of:

(i) a glucoamylase comprising the polypeptide of SEQ ID NO: 1 or SEQ ID NO: 2;

(ii) a glucoamylase comprising an amino acid sequence having at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 1 or SEQ ID NO: 2.

In another embodiment the glucoamylase is the Gloeophyllum trabeum glucoamylase shown in SEQ ID NO: 1 having one of the following substitutions: V59A; S95P; A121P; T119W; S95P+A121P; V59A+S95P; S95P+T119W; V59A+S95P+A121P; or S95P+T119W+A121P, especially S95P+A121P; and wherein the glucoamylase has at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 1.

In another embodiment the glucoamylase is selected from from the group consisting of:

(i) a glucoamylase comprising the polypeptide of SEQ ID NO: 3;

(ii) a glucoamylase comprising an amino acid sequence having at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 3.

In another embodiment the glucoamylase is selected from the group consisting of:

(i) a glucoamylase comprising the polypeptide of SEQ ID NO: 4;

(ii) a glucoamylase comprising an amino acid sequence having at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 4.

According to another preferred embodiment of the processes of the invention the yeast fermenting organism expresses an alpha-amylase, particularly the alpha-amylase expressed from the fermenting organism is a Rhizomucor alpha-amylase, particularly a Rhizomucor pusillus alpha-amylase, or an Aspergillus alpha-amylase, particularly an Aspergillus terreus alpha-amylase.

In one embodiment of the process of the invention the alpha-amylase is Rhizomucor pusillus alpha-amylase with an Aspergillus niger glucoamylase linker and starch-binding domain (SBD) as shown in SEQ ID NO: 5, preferably one having at least one of the following substitutions or combinations of substitutions: D165M; Y141W; Y141R; K136F; K192R; P224A; P224R; S123H+Y141W; G2OS+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C, especially G128D+D143N (using SEQ ID NO: 5 for numbering), and wherein the alpha-amylase has at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 5.

In one embodiment of the process of the invention the alpha-amylase is an Aspergillus terreus alpha-amylase selected from the group consisting of:

(i) an alpha-amylase comprising the polypeptide of SEQ ID NO: 6;

(ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 6.

In a preferred embodiment of the process according to the invention the glucoamylase is the Trametes cingulata glucoamylase shown in SEQ ID NO: 3 and the alpha-amylase is Rhizomucor pusillus alpha-amylase with an Aspergillus niger glucoamylase linker and starch-binding domain (SBD) shown in SEQ ID NO: 5.

In another preferred embodiment of the process according to the invention the glucoamylase is the Gloeophyllum abietinum glucoamylase shown in SEQ ID NO: 2 and the alpha-amylase is is Rhizomucor pusillus alpha-amylase with an Aspergillus niger glucoamylase linker and starch-binding domain (SBD) shown in SEQ ID NO: 5, preferably one having the following substitutions G128D+D143N (using SEQ ID NO: 5 for numbering).

In another preferred embodiment of the process according to the invention the glucoamylase is the Pycnoporus sanguineus glucoamylase shown in SEQ ID NO: 4 herein, and the alpha-amylase is the Rhizomucor pusillus with an Aspergillus niger glucoamylase linker and starch-binding domain (SBD), preferably the one disclosed as SEQ ID NO: 5, preferably one having one or more of the following substitutions: G128D, D143N, especially G128D+D143N.

In an embodiment the fermenting organism used in a process of the invention, in particular MBG4911, expresses glucoamylase, in particular the one shown in SEQ ID NO: 4, or an alpha-amylase having at least 80%, at least 90%, at least 95%, at least 97%, at least 99% sequence identity to SEQ ID NO: 4.

In a specific embodiment the strain is the AgJg013 (see Examples 4 and 5) or a strain having properties that are about the same as that of AgJg013.

In an embodiment the fermenting organism used in a process of the invention, in particular MBG4911, expresses alpha-amylase, in particular the one shown in SEQ ID NO: 5, in particular one further comprising G128D+D143N substitutions, or an alpha-amylase having at least 80%, at least 90%, at least 95%, at least 97%, at least 99% sequence identity to SEQ ID NO: 5.

In a specific embodiment the strain is MLBA795 (see Example 6) or a strain having properties that are about the same as that of MLBA795.

In another embodiment the fermenting organism used in a process of the invention, in particular MBG4911, expresses:

a glucoamylase, in particular the one shown in SEQ ID NO: 4 or a glucoamylase having at least 80%, at least 90%, at least 95%, at least 97% or at least 99% sequence identity to SEQ ID NO: 4; and

an alpha-amylase, in particular the one shown in SEQ ID NO: 5, especially one further comprising G128D+D143N substitutions, or an alpha-amylase having at least 80%, at least 90%, at least 95%, at least 97%, at least 99% sequence identity to SEQ ID NO: 5.

In a specific embodiment the strain is MLBA821 (see Example 8) or a strain having properties that are about the same as that of MLBA821.

### Starch-Containing Materials

According to the invention sugars may be derived from starch-containing materials. Any suitable starch-containing starting material, including granular starch, may be used according to the present invention. The starting material is generally selected based on the desired fermentation product. Examples of starch-containing starting materials, suitable for use in a process of present invention, include whole grains, corns, wheat, barley, rye, milo, sago, cassava, tapioca, sorghum, rice, peas, beans, and sweet potatoes, or mixtures thereof, or cereals, or sugar-containing raw materials, such as molasses, fruit materials, sugar cane or sugar beet, potatoes. Contemplated are both waxy and non-waxy types of corn and barley.

The term “granular starch” means raw uncooked starch, i.e., starch in its natural form found in cereal, tubers or grains. Starch is formed within plant cells as tiny granules insoluble in water. When put in cold water, the starch granules may absorb a small amount of the liquid and swell. At temperatures up to 50° C. to 75° C. the swelling may be reversible. However, with higher temperatures an irreversible swelling called “gelatinization” begins. Granular starch to be processed may in an embodiment be a highly refined starch, preferably at least 90%, at least 95%, at least 97% or at least 99.5% pure, or it may be a more crude starch containing material comprising milled whole grain including non-starch fractions such as germ residues and fibers. The raw material, such as whole grain, is milled in order to open up the structure and allowing for further processing. Two milling processes are preferred according to the invention: wet and dry milling. In dry milling whole kernels are milled and used. Wet milling gives a good separation of germ and meal (starch granules and protein) and is often applied at locations where the starch hydrolyzate is used in production of syrups. Both dry and wet milling is well known in the art of starch processing and is equally contemplated for the process of the invention.

The starch-containing material may be reduced in particle size, preferably by dry or wet milling, in order to expose more surface area. In an embodiment the particle size is between 0.05 to 3.0 mm, preferably 0.1-0.5 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen.

The present invention may be further described by the following numbered paragraphs:

Paragraph [1]: A process of producing ethanol from starch-containing material comprising:

(a) saccharifying the starch-containing material; and

(b) fermenting using a fermentation organism;

wherein

saccharification and/or fermentation is done in the presence of at least a glucoamylase and optionally an alpha-amylase;

the fermenting organism is Saacaromyces cerevisiae;

and wherein a glucoamylase and/or an alpha-amylase is expressed from the fermenting organism.

Paragraph [2]: The process according to paragraph [1], wherein the starch containing material is either gelatinized or ungelatinized starch.

Paragraph [3]: The process according to paragraph [2], wherein a liquefaction step precedes the saccharification step, and wherein the liquefaction step is performed in the presence of at least a bacterial alpha-amylase, such as an alpha-amylase from Bacillus sp., particularly Bacillus stearothermophilus.

Paragraph [4]: The process according to any of paragraphs [1]-[3], wherein the Saccharomyces cerevisiae is MBG4931 (deposited under Accession No. V15/004036 at National Measurement Institute, Victoria, Australia) or a fermenting organism having properties that are about the same as that of Saacaromyces cerevisiae MBG4931 or a derivative of Saacaromyces strain V15/004036 having defining characteristics of strain V15/004036.

Paragraph [5]: The process according to any of paragraphs [1]-[3], wherein the Saccharomyces cerevisiae is MBG4851 (deposited under Accession No. V14/004037 at National Measurement Institute, Victoria, Australia) or a fermenting organism strain having properties that are about the same as that of Saacaromyces cerevisiae MBG4851, or a derivative of Saacaromyces strain V14/004037 having the defining characteristics of strain V14/004037.

Paragraph [6]: The process according to any of paragraphs [1]-[3], wherein the Saccharomyces cerevisiae is MBG4911 (deposited as V15/001459 at National Measurement Institute, Victoria, Australia) or a fermenting organism strain having properties that are about the same as that of Saacaromyces cerevisiae MBG4911 or a derivative of Saacaromyces strain V15/001459 having defining characteristics of strain V15/001459.

Paragraph [7]: The process according to any of paragraphs [1]-[3], wherein the Saccharomyces cerevisiae is MBG4913 (deposited as V15/001460 at National Measurement Institute, Victoria, Australia) or a fermenting organism strain having properties that are about the same as that of Saacaromyces cerevisiae is MBG4913 or a derivative of Saacaromyces strain V15/001460 having defining characteristics of strain V15/001460.

Paragraph [8]: The process according to any of paragraphs [1]-[3], wherein the Saccharomyces cerevisiae is MBG4914 (deposited as V15/001461 at National Measurement Institute, Victoria, Australia) or a fermenting organism strain having properties that are about the same as that of Saacaromyces cerevisiae is MBG4914 or a derivative of Saacaromyces strain V15/001461 having defining characteristics of strain V15/001461.

Paragraph [9]: The process of paragraphs [1]-[8], wherein the glucoamylase is expressed from the fermenting organism and is a Gloeophyllum glucoamylase, preferably Gloeophyllum trabeum, Gloeophyllum sepiarium, or Gloeophyllum abietinum glucoamylase.

Paragraph [10]: The process of paragraph [9], wherein the glucoamylase is selected from the group consisting of:

(i) a glucoamylase comprising the polypeptide of SEQ ID NO: 1 or SEQ ID NO: 2;

(ii) a glucoamylase comprising an amino acid sequence having at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 1 or SEQ ID NO: 2.

Paragraph [11]: The process of paragraph [9] or [10], wherein the glucoamylase is the Gloeophyllum trabeum glucoamylase shown in SEQ ID NO: 1 having one of the following substitutions: V59A; S95P; A121P; T119W; S95P+A121P; V59A+S95P; S95P+T119W; V59A+S95P+A121P; or S95P+T119W+A121P, especially S95P+A121P.

Paragraph [12]: The process of any of paragraphs [1]-[8], wherein the glucoamylase is expressed from the fermenting organism and is a Trametes glucoamylase, preferably a Trametes cingulata glucoamylase.

Paragraph [13]: The process of paragraph [12], wherein the glucoamylase is selected from the group consisting of:

(i) a glucoamylase comprising the polypeptide of SEQ ID NO: 3;

(ii) a glucoamylase comprising an amino acid sequence having at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 3.

Paragraph [14]: The process of any of paragraphs [1]-[8], wherein the glucoamylase is expressed from the fermenting organism and is a Pycnoporus glucoamylase, particularly Pycnoporus sanguineus glucoamylase.

Paragraph [15]: The process of paragraph [14], wherein the glucoamylase is selected from the group consisting of:

(i) a glucoamylase comprising the polypeptide of SEQ ID NO: 4;

(ii) a glucoamylase comprising an amino acid sequence having at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 4.

Paragraph [16]: The process of any of paragraphs [1]-[15], wherein the alpha-amylase is expressed from the fermenting organism and is derived from Rhizomucor pusillus or Aspergillus terreus.

Paragraph [17]: The process of paragraph [16], wherein the alpha-amylase is Rhizomucor pusilus alpha-amylase with an Aspergillus niger glucoamylase linker and starch-binding domain (SBD) as shown in SEQ ID NO: 5, preferably one having at least one of the following substitutions or combinations of substitutions: D165M; Y141W; Y141R; K136F; K192R; P224A; P224R; S123H+Y141W; G20S +Y141W; A76G +Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C, especially G128D+D143N (using SEQ ID NO: 5 for numbering), and wherein the alpha-amylase has at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 5.

Paragraph [18]: The process of paragraph [16], wherein the alpha-amylase is Aspergillus terreus alpha-amylase selected from the group consisting of:

(i) an alpha-amylase comprising the polypeptide of SEQ ID NO: 6;

(ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 6.

Paragraph [19]: The process of any of paragraphs [1]-[18], wherein the glucoamylase and alpha-amylase are expressed from the fermenting organism, wherein the glucoamylase is the Trametes cingulata glucoamylase shown in SEQ ID NO: 3 and wherein the alpha-amylase is Rhizomucor pusillus alpha-amylase with an Aspergillus niger glucoamylase linker and starch-binding domain (SBD) shown in SEQ ID NO: 5.

Paragraph [20]: The process any of paragraphs [1]-[18], wherein the glucoamylase and alpha-amylase are expressed from the fermenting organism, wherein the glucoamylase is the Gloeophyllum abietinum glucoamylase shown in SEQ ID NO: 2 and wherein the alpha-amylase is a Rhizomucor pusillus alpha-amylase with an Aspergillus niger glucoamylase linker and starch-binding domain (SBD) shown in SEQ ID NO: 5, preferably one having the following substitutions G128D+D143N (using SEQ ID NO: 5 for numbering).

Paragraph [21]: The process of any of paragraphs [1]-[18], wherein the glucoamylase and alpha-amylase are expressed from the fermenting organism, wherein the glucoamylase is the Pycnoporus sanguineus glucoamylase shown in SEQ ID NO: 4, and wherein the alpha-amylase is the Rhizomucor pusillus with an Aspergillus niger glucoamylase linker and starch-binding domain (SBD), preferably the one disclosed as SEQ ID NO: 5, preferably one having one or more of the following substitutions: G128D, D143N, especially G128D+D143N.

Paragraph [22]: The process of paragraphs [1]-[21], wherein the fermenting organism, in particular MBG4911, expresses glucoamylase, in particular the one shown in SEQ ID NO: 4 or a glucoamylase having at least 80%, at least 90%, at least 95%, at least 97%, at least 99% sequence identity to SEQ ID NO: 4.

Paragraph [23]: The process of paragraphs [1]-[21], wherein the fermenting organism, in particular MBG4911, expresses alpha-amylase, in particular the one shown in SEQ ID NO: 5, in particular one compriing G128D+D143N substitutions, or an alpha-amylase having at least 80%, at least 90%, at least 95%, at least 97%, at least 99% sequence identity to SEQ ID NO: 5.

Paragraph [24]: The process of paragraphs [1]-[22], wherein the fermenting organism, in particular MBG4911, expresses

a glucoamylase, in particular the one shown in SEQ ID NO: 4 or a glucoamylase having at least 80%, at least 90%, at least 95%, at least 97% or at least 99% sequence identity to SEQ ID NO: 4;

an alpha-amylase, in particular the one shown in SEQ ID NO: 5, in particular one comprising G128D+D143N substitutions, or an alpha-amylase having at least 80%, at least 90%, at least 95%, at least 97%, at least 99% sequence identity to SEQ ID NO: 5.

Paragraph [25]: A yeast strain comprising one or more expression constructs encoding a glucoamylase and/or an alpha-amylase, wherein the yeast is derived from a parent strain selected from MBG4851, MBG4931, MBG4911, MBG4913and MBG4914; and wherein the glucoamylase is selected from glucoamylases obtainable from Gloeophyllum, Pycnoporous, or Trametes.

Paragraph [26]: The yeast strain according to paragraph [25], wherein the glucoamylase is selected from a Gloeophyllum trabeum, Gloeophyllum sepiarium, or Gloeophyllum abietinum glucoamylase.

Paragraph [27]: The yeast strain of paragraph [26], wherein the glucoamylase is selected from the group consisting of:

(i) a glucoamylase comprising the polypeptide of SEQ ID NO: 1 or SEQ ID NO: 2;

(ii) a glucoamylase comprising an amino acid sequence having at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 1 or SEQ ID NO: 2.

Paragraph [28]: The yeast strain of any of paragraphs [25]-[27], wherein the glucoamylase is the Gloeophyllum trabeum glucoamylase shown in SEQ ID NO: 1 having one of the following substitutions: V59A; S95P; A121P; T119W; S95P+A121P; V59A+S95P; S95P+T119W; V59A+S95P+A121P; or S95P+T119W+A121P, especially S95P+A121P.

Paragraph [29]: The yeast strain of paragraph [25], wherein the glucoamylase is selected from a Trametes cingulata glucoamylase.

Paragraph [30]: The yeast strain of paragraph [29], wherein the glucoamylase is selected from the group consisting of:

(i) a glucoamylase comprising the polypeptide of SEQ ID NO: 3;

(ii) a glucoamylase comprising an amino acid sequence having at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 3.

Paragraph [31]: The yeast strain of paragraph [25], wherein the glucoamylase is selected from a Pycnoporus sanguineus glucoamylase.

Paragraph [32]: The yeast strain of paragraph [31], wherein the glucoamylase is selected from the group consisting of:

(i) a glucoamylase comprising the polypeptide of SEQ ID NO: 4;

(ii) a glucoamylase comprising an amino acid sequence having at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 4.

Paragraph [33]: A yeast strain comprising one or more expression constructs encoding a glucoamylase and/or an alpha-amylase, wherein the yeast is derived from a parent strain selected from MBG4851, MBG4931, MBG4911, MBG4913 and MBG4914; and wherein the alpha-amylase is selected from a Rhizomucor pusillus or Aspergillus terreus alpha-amylase.

Paragraph [34]: The yeast strain of paragraph [33], wherein the alpha-amylase is Rhizomucor pusillus alpha-amylase with an Aspergillus niger glucoamylase linker and starch-binding domain (SBD) as shown in SEQ ID NO: 5, preferably one having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C, especially G128D+D143N (using SEQ ID NO: 5 for numbering), and wherein the alpha-amylase has at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 5.

Paragraph [35]: The yeast strain of paragraph [33], wherein the alpha-amylase is Aspergillus terreus alpha-amylase selected from the group consisting of:

(i) an alpha-amylase comprising the polypeptide of SEQ ID NO: 6;

(ii) an alpha-amylase comprising an amino acid sequence having at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 6.

Paragraph [36]: The yeast strain according to any of the paragraphs [25]-[35], wherein the yeast is derived from a parent strain MBG4931.

Paragraph [37]: The yeast strain according to any of the paragraphs [25]-[35], wherein the yeast is derived from a parent strain MBG4911.

Paragraph [38]: The yeast strain according to any of the paragraphs [25]-[35], wherein the yeast is derived from a parent strain MBG4931.

Paragraph [39]: The yeast according to any of paragraphs [25]-[38], wherein the parent yeast strain expresses the glucoamylase shown in SEQ ID NO: 4, or a glucoamylase having at least 80%, at least 90%, at least 95%, at least 97% or at least 99% sequence identity to SEQ ID NO: 4.

Paragraph [40]: The yeast according to paragraph [39], wherein the parent yeast strain is MBG4911.

Paragraph [41]: The yeast according to paragraph [39] or [40], wherein the yeast strain is strain AgJg013 or a strain having properties which are about the same as that of AgJg013.

Paragraph [41]: The yeast according to any of paragraphs [25]-[41], wherein the parent yeast strain expresses the alpha-amylase shown in SEQ ID NO: 5, in particular one further comprising G128D+D143N substitutions, or an alpha-amylase having at least 80%, at least 90%, at least 95%, at least 97% or at least 99% sequence identity to SEQ ID NO: 5.

Paragraph [42]: The yeast according to paragraph [41], wherein the parent yeast strain is MBG4911.

Paragraph [43]: The yeast according to paragraph [40] or [41], wherein the yeast strain is strain MLBA795 or a strain having properties which are about the same as that of MBGMLBA795.

Paragraph [44]: The yeast according to any of paragraphs [25]-[43], wherein the parent yeast strain expresses:

the alpha-amylase shown in SEQ ID NO: 5, in particular one further comprising G128D+D143N substitutions, or an alpha-amylase having at least 80%, at least 90%, at least 95%, at least 97% or at least 99% sequence identity to SEQ ID NO: 5; and

the glucoamylase shown in SEQ ID NO: 4, or a glucoamylase having at least 80%, at least 90%, at least 95%, at least 97% or at least 99% sequence identity to SEQ ID NO: 4.

Paragraph [45]: The yeast according to paragraph [44], wherein the parent yeast strain is MBG4911.

Paragraph [46]: The yeast according to paragraph [44] or [45], wherein the yeast strain is strain MLBA821 or a strain having properties that are about the same as that of MLBA821.

### Methods and Assays

Percent Identity: The relatedness between two amino acid sequences or between two polynucleotide sequences is described by the parameter “identity”.

For purposes of the present invention, the degree of identity between two amino acid sequences is determined by the Clustal method (Higgins, 1989, CAB/OS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=1, gap penalty=3, windows=5, and diagonals=5.

For purposes of the present invention, the degree of identity between two polynucleotide sequences is determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

### Enzymes:

GsAMG: Glucoamylase derived from Gloeophyllum sepiarium (formally known Gloeophyllum abietinum) disclosed in SEQ ID NO: 2 herein.

GtAMG: Glucoamylase derived from Gloeophyllum trabeum disclosed in SEQ ID NO: 1 herein.

PsAMG: Glucoamylase derived from Pycnoporus sanguineus disclosed as shown in SEQ ID NO: 4 in WO 2011/066576 and in SEQ ID NO: 4 herein.

TcAMG: Glucoamylase derived from Trametes cingulata shown in SEQ ID NO: 3 herein or SEQ ID NO: 2 in WO 2006/69289.

JA126: Alpha-amylase derived from Rhizomucor pusillus with an Aspergillus niger glucoamylase linker and starch-binding domain (SBD) shown in SEQ ID NO: 5 herein.

PE096: Alpha-amylase derived from Rhizomucor pusillus with an Aspergillus niger glucoamylase linker and starch-binding domain (SBD) shown in SEQ ID NO: 5 herein, with the following substitutions: G128D+D143N.

AtAA: Alpha-amylase derived from Aspergillus terreus shown in SEQ ID NO: 6 herein.

Glucoamylase blend A: Blend comprising Talaromyces emersonii glucoamylase disclosed as SEQ ID NO: 34 in WO99/28448, Trametes cingulata glucoamylase disclosed as SEQ ID NO: 3 in WO 06/69289, and Rhizomucor pusillus alpha-amylase with Aspergillus niger glucoamylase linker and starch binding domain (SBD) disclosed in SEQ ID NO: 5 herein having the following substitutions G128D+D143N using SEQ ID NO: 5 numbering (activity ratio in AGU:AGU:FAU-F is about 29:8:1).

LACTROL® is a dry antimicrobial formulation of Virginiamycin and dextrose.

### Glucoamylase Activity

Glucoamylase activity may be measured in Glucoamylase Units (AGU).

### Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

TABLE 1
AMG incubation conditions

| Substrate: | maltose 23.2 mM | |
|---|---|---|
| Buffer: | acetate 0.1M | |
| pH: | 4.30 ± 0.05 | |
| Incubation temperature: | 37° C. ± 1 | |
| Reaction time: | 5 | minutes |
| Enzyme working range: | 0.5-4.0 | AGU/mL |

TABLE 2
Color reaction conditions

| GlucDH: | 430 | U/L |
|---|---|---|
| Mutarotase: | 9 | U/L |
| NAD: | 0.21 | mM |
| Buffer: | phosphate 0.12M; 0.15M NaCl | |
| pH: | 7.60 ± 0.05 | |
| Incubation temperature: | 37° C. ± 1 | |
| Reaction time: | 5 | minutes |
| Wavelength: | 340 | nm |

A folder (EB-SM-0131.02/01) describing this analytical method in more detail is available on request from Novozymes A/S, Denmark, which folder is hereby included by reference.

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

### Acid Alpha-Amylase Activity

When used according to the present invention the activity of an acid alpha-amylase may be measured in AFAU (Acid Fungal Alpha-amylase Units) or FAU-F.

### Acid Alpha-Amylase Activity (AFAU)

Acid alpha-amylase activity may be measured in AFAU (Acid Fungal Alpha-amylase

Units), which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

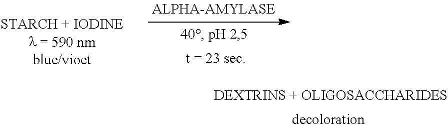

TABLE 3
Standard conditions/reaction conditions.

| Substrate: | Soluble starch, approx. 0.17 g/L | |
|---|---|---|
| Buffer: | Citrate, approx. 0.03M | |
| Iodine (I2): | 0.03 | g/L |
| CaCl₂: | 1.85 | mM |
| pH: | 2.50 ± 0.05 | |
| Incubation temperature: | 40° | C. |
| Reaction time: | 23 | seconds |
| Wavelength: | 590 | nm |
| Enzyme concentration: | 0.025 | AFAU/mL |
| Enzyme working range: | 0.01-0.04 | AFAU/mL |

A folder EB-SM-0259.02/01 describing this analytical method in more detail is available upon request to Novozymes A/S, Denmark, which folder is hereby included by reference.

### Determination of FAU-F

FAU-F Fungal Alpha-Amylase Units (Fungamyl) is measured relative to an enzyme standard of a declared strength.

TABLE 4
Reaction conditions

| Temperature | 37° | C. |
|---|---|---|
| pH | 7.15 | |
| Wavelength | 405 | nm |
| Reaction time | 5 | min |
| Measuring time | 2 | min |

A folder (EB-SM-0216.02) describing this standard method in more detail is available on request from Novozymes A/S, Denmark, which folder is hereby included by reference.

### Deposit of Biological Material

The following biological material has been deposited under the terms of the Budapest Treaty with the National Measurement Institute, 1/153 Bertie Street, Port Melbourne, Victoria 3207, Australia and given the following accession number:

| Deposit | Accession Number | Date of Deposit |
|---|---|---|
| MBG4851 | V14/004037 | Feb. 17, 2014 |
| MBG4911 | V15/001459 | Jan. 13, 2015 |
| MBG4913 | V15/001460 | Jan. 13, 2015 |
| MBG4914 | V15/001461 | Jan. 13, 2015 |
| MBG4931 | V15/004036 | Feb. 19, 2015 |

The strains above have been deposited under conditions that assure that access to the culture will be available during the pendency of this patent application to one determined by foreign patent laws to be entitled thereto. The deposit represents a substantially pure culture of the deposited strain. The deposit is available as required by foreign patent laws in countries wherein counterparts of the subject application or its progeny are filed. However, it should be understood that the availability of a deposit does not constitute a license to practice the subject invention in derogation of patent rights granted by governmental action.

### EXAMPLES

### Example 1: Construction of Yeast Strains Expressing Alpha-Amylase (AA), Qlucoamylase (AMG), or an Alpha-Amylase (AA)+a Glucoamylase (AMG)

### Construction of Homozygous Strains Via Two-Step Transformation

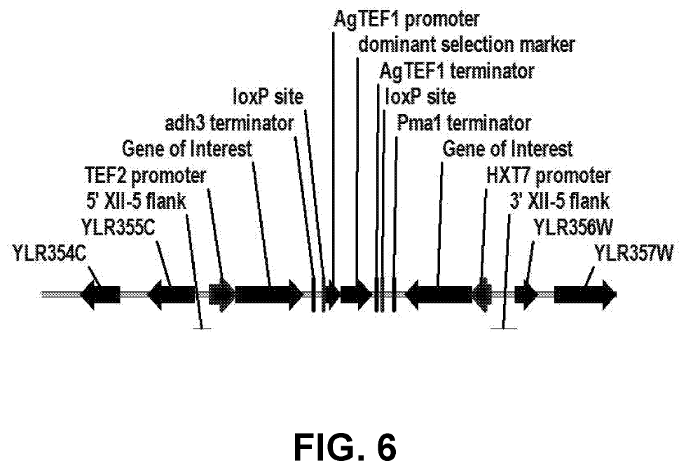

Expression cassettes for the desired genes were targeted to the XI-1 or XII-5 integration sites as described in Mikkelsen et al. (Metabolic Engineering v14 (2012) pp104-111). Two plasmids employing a split-marker approach were used for each integration event, each containing an expression cassette and approximately two-thirds of a dominant selection marker. The left-hand plasmid contained 5′ flanking DNA homologous to the desired integration site, the S. cerevisiae TEF2 promoter driving expression of the gene of interest codon-optimized for expression in S. cerevisiae, the S. cerevisiae ADH3 terminator, a loxP site, and the 5′ two-thirds of a dominant selection marker under control of the Ashbya gossypii TEF1 promoter. The right-hand plasmid contains the 3′ two-thirds of the dominant selection marker with the Ashbya gossypii TEF1 terminator, a loxP site, an expression cassette in the reverse orientation relative to the dominant selection marker composed of the S. cerevisiae HXT7 promoter driving expression of the gene of interest codon-optimized for expression in S. cerevisiae with the S. cerevisiae PMA1 terminator, and 3′ flanking DNA homologous to the desired integration site. A left-hand and right-hand plasmid pair was linearized with restriction enzymes and transformed into S. cerevisiae strain MBG4931 using lithium acetate transformation (see Gietz and Woods, 2006, Methods in Molecular Biology, v 313 pp107-120). Since MBG4931 is a diploid yeast, the desired integration construct was first integrated using kanamycin resistance as the dominant selection marker, followed by PCR screening to confirm the desired integration event. A confirmed heterozygous transformant was then transformed again using an expression cassette pair with the nourseothricin resistance marker. PCR screening was used to confirm homozygous modification of the targeted chromosomal integration site. A diagram of the expression cassette at one chromosome of the XII-5 integration site is shown in FIG. 6. Resulting strains yMHCT390, yMHCT392, yMHCT394, yMHCT396 and MEJ1697 are shown in Table 5 below with corresponding expressed glucoamylase and/or alpha-amylase of interest and integration locus.

Expression cassettes for the desired genes were targeted to the XI-1 (PE096) or XII-5 (PsAMG integration sites as described in Mikkelsen et al. (Metabolic Engineering v14 (2012) pp104-111). Two plasmids employing a split-marker approach were used for each integration event, each containing an expression cassette and approximately two-thirds of a dominant selection marker. The left-hand plasmid contained 5′ flanking DNA homologous to the desired integration site, the S. cerevisiae TEF2 promoter driving expression of the gene of interest codon-optimized for expression in S. cerevisiae, the S. cerevisiae ADH3 terminator, a loxP site, and the 5′ two-thirds of a dominant selection marker under control of the Ashbya gossypii TEF1 promoter. The right-hand plasmid contains the 3′ two-thirds of the dominant selection marker with the Ashbya gossypii TEF1 terminator, a loxP site, an expression cassette in the reverse orientation relative to the dominant selection marker composed of the S. cerevisiae HXT7 promoter driving expression of the gene of interest codon-optimized for expression in S. cerevisiae with the S. cerevisiae PMA1 terminator, and 3′ flanking DNA homologous to the desired integration site. A left-hand and right-hand plasmid pair containing the AA variant PE096 expression cassettes targeting to XI-1 was linearized with restriction enzymes and transformed into S. cerevisiae strain MBG4911 using lithium acetate transformation (see Gietz and Woods, 2006, Methods in Molecular Biology, v 313 pp107-120). Since MBG4911 is a diploid yeast, the desired integration construct was first integrated using kanamycin resistance as the dominant selection marker, followed by PCR screening to confirm the desired integration event. A confirmed heterozygous transformant was then transformed again using an expression cassette pair with the nourseothricin resistance marker. PCR screening was used to confirm homozygous modification of the XI-1 integration site creating strain MlBa787.

The antibiotic markers present in MlBa787 are flanked by loxP sites. MlBa787 was transformed with plasmid pFYD80 that includes a gene encoding the CRE recombinase, a site specific enzyme that facilitates recombination between neighboring loxP sites (Güldener et al., 2002). Plasmid pFYD80 is maintained as a non-integrative, free replicating molecule. This approach enables the specific excision of both selective markers. MlBa787 was transformed with plasmid pFYD80, and transformants were selected on plates containing zeocin. Zeocin resistance is encoded in pFYD80. Subsequently, screening for transformants that have lost nourseothricin and kanamycin resistance was performed. Sensitive strains were grown in YPD liquid until loss of pFYD80 plasmid was obtained. Strain MlBa795 was selected and shown to be zeocin sensitive as a result of the loss of plasmid pFYD80.

The S. cerevisiae strain MlBa795 expresses the AA variant PE096 at the XI-1 integration site. MlBa795 was modified to express the Ps glucoamylase at the XII-5 integration site. The Ps glucoamylase expression cassettes were introduced into MlBa795 as described above for the AA variant PE096. This resulted in strain MlBa821 which contains Ps glucoamylase expression cassettes at XII-5 and the AA variant PE096 expression cassettes at XI-1. MlBa821 still contains the antibiotic selection markers at the XII-5 integration site and therefore is resistant to kanamycin and nourseothricin. The antibiotic markers were removed from MlBa821 by transformation with pFYD80 as described above. The resulting antibiotic marker free strain is called MlBa855 and contains 4 copies of the AA variant PE096 at XI-1 and 4 copies of the Ps glucoamylase at XII-5. Resulting strains MLBA787, MLBA795, MLBA821, and MLBA855 are shown in Table 5 below with corresponding expressed glucoamylase and/or alpha-amylase of interest and integration locus.

Additional examples of homozygous strains constructed via two-step transformation are shown below in Table 5.

### Construction Homozygous Strains Via One-Step Transformation

To simultaneously modify both alleles of a target locus in a diploid strain in a single transformation, two left-hand and right-hand plasmid pairs were used, one pair with the kanamycin resistance marker and the other pair with the nourseothricin marker (as described supra). Both pairs were linearized with restriction enzymes and all four resulting linearized DNAs were simultaneously transformed into an S. cerevisiae host strain using electroporation (see Thompson, et al., YEAST vol. 14: 565-71, 1998). The transformation mixture was plated to a plate containing both kanamycin and nourseothricin, and colonies resistant to both selection agents were PCR screened to confirm homozygous modification of the targeted chromosomal integration site. A confirmed transformant was thus homozygous for the expression cassette at the target locus, with one chromosome containing the kanamycin marker and the other chromosome containing the nourseothricin marker. For some strains, marker removal was subsequently carried out using the plasmid pFYD80 that includes a gene encoding the CRE recombinase as described supra.

Examples of homozygous strains constructed via one-step transformation are shown below in Table 5.

TABLE 5
AA and AMG-expressing yeast strains

| | | | | copies of | antibiotic re- | |
|---|---|---|---|---|---|---|
| | | integra- | expressed | expressed | sistance mark- | |
| Strain | background | tion locus | protein | protein | ers | construction method |
| yMHCT390 | MBG4931 | XI-1 | AtAA | 4 | kanamycin and | two-step |
| | | | | | nourseothricin | |
| yMHCT392 | MBG4931 | XII-5 | AtAA | 4 | kanamycin and | two-step |
| | | | | | nourseothricin | |
| yMHCT394 | MBG4931 | XI-1 | PE096 AA | 4 | kanamycin and | two-step |
| | | | | | nourseothricin | |
| yMHCT396 | MBG4931 | XII-5 | PE096 AA | 4 | kanamycin and | two-step |
| | | | | | nourseothricin | |
| MEJI697 | MBG4931 | XII-5 | GsAMG | 4 | none | two-step + pFYD80 |
| MEJI705 | MBG4931 | XII-5 | GsAMG | 4 | none | two-step + pFYD80 |
| AgJg013 | MBG4911 | XII-5 | PsAMG | 4 | kanamycin and | two-step |
| | | | | | nourseothricin | |
| MLBA787 | MBG4911 | XI-1 | PE096 AA | 4 | kanamycin and | two-step |
| | | | | | nourseothricin | |
| MLBA795 | MBG4911 | XI-1 | PE096 AA | 4 | none | two-step, pFYD80 |
| MLBA821 | MBG4911 | XI-1, XII-5 | PE096 AA, | 4, 4 | kanamycin and | two-step + pFYD80 at XI-1; |
| | | | PsAMG | | nourseothricin | two-step at XII-5 |
| MLBA855 | MBG4911 | XI-1, XII-5 | PE096 AA, | 4, 4 | none | two-step + pFYD80 at XI-1; |
| | | | PsAMG | | | two-step at XII-5 + pFYD80 |
| MLBA888 | Ethanol Red | XI-1, XII-5 | PE096 AA, | 4, 4 | kanamycin and | two-step + pFYD80 at XII-5; |
| | | | PsAMG | | nourseothricin | one-step at XI-1 + pFYD80 |
| MLBA889 | MBG4931 | XI-1, XII-5 | PE096 AA, | 2, 6 | none | two-step + pFYD80 at XII-5; |
| | | | GsAMG | | | one-step at XI-1 + pFYD80 |
| MLBA891 | MBG4931 | XI-1, XII-5 | PE096 AA, | 4, 4 | none | two-step + pFYD80 at XII-5; |
| | | | GsAMG | | | one-step at XI-1 + pFYD80 |
| PsinER1 | Ethanol Red | XII-5 | PsAMG | 4 | none | two-step + pFYD80 |
| MHCT408 | MBG4931 | XII-5 | GtAMG | 4 | none | two-step + pFYD80 |

### Example 2: Application Performance of a Yeast Expressing a Fungal Alpha Amylases in a Raw Starch Ethanol Process (MBG4931 Expressing AA)

The following experiments were performed using ground corn. Urea was supplemented to 500 ppm.

Experiment 1 was performed at bottle scale. 150 grams of corn mash was added to a 250 ml Wheaton bottle. All strains were dosed at 0.5 AGU/gDS using glucoamylase from Pycnoporus sanguineus (PsAMG/SEQ ID NO: 4). A fungal alpha-amylase variant, PE096, a Rhizomucor pusillus alpha-amylase with an Aspergillus niger glucoamylase linker and starchbinding domain (SBD) as shown in SEQ ID NO: 5, having substitutions G128D+D143N, was used as the exogenous alpha-amylase in this experiment. The parent strain was dosed with

PE096 at 0.031, 0.016, or 0.008 FauF/ gDS (16, 32, and 64 ratios respectively). The yeast strains used as the fermenting organism expressed a fungal alpha-amylase selected as either PE096 (strains MHCT394 and MHCT396) or a wild type alpha-amylase derived from Aspergillus terreus (SEQ ID NO: 6) (strains MHCT 390 and MHCT 392). Alpha-amylase expressing strains were all tested at the 64 ratio.

Experiment 2 was performed at tube scale, with approximately 5 grams of corn mash in a 15 ml conical tube. The parent strain was dosed at the 16, 32, or 64 ratio, glucoamylase (GA) only, or no exogenous enzyme added. The Alpha-amylase (AA) expressing strains were only tested under the GA only or no exogenous enzyme added conditions. When GA was added, a dose of 0.5 AGU/gDS PsAMG was used.

### Results:

Experiment 1: At 55 hours the AA expressing strains at the 64 ratio outperformed the parent strain at all 3 ratios. At 73 hours of fermentation, the Alpha Amylase expressing strains, dosed at a RSH ratio of 64 have statistically identical performance to the parent strain dosed at a RSH ratio of 16. This indicates that 75% alpha amylase reduction is possible.

Experiment 2: At 72 hours, the AA expressing strains with only GA added were able to match the performance of the parent strain at a RSH16 ratio, compared to the parent which only generated ˜49% of the total ethanol under these conditions. This suggests that it is possible to remove most, if not all of the AA from the enzyme mixture when using these AA expressing strains. When no exogenous enzyme is added, the strains are able to generate ˜75% of the ethanol generated by the full enzyme mixture in the parent strain, whereas the parent strain generates less 2% of the total ethanol under these conditions.

### Example 3: Application Performance of a Yeast Expressing a Glucoamylase (AMG) in an SSF Process on Liquefied Starch (MBG4931 Expressing AMG)

A liquefied mash prepared using a commercial bacterial alpha-amylase product, Liquozyme SODS, was used to prepare the liquefied mash. Mash solids were read to be 33.76% using a moisture balance. Approximately 5 grams of corn mash was fermented in pre-weighed 15 ml flip top conical tubes with a small hole drilled for gas release. A saccharification composition, glucoamylase blend A dosed at 0.6 AGU/gDS for the parent strain (MBG4931) as the full enzyme dose. The parent strain (MBG4931), the glucoamylase producing strain producing the Gloeophyllum sepiarium glucoamylase (GsAMG/SEQ ID NO: 2) (MeJi697) and Transferm Yield+ were all dosed at 0.3 AGU/gDS to test a 50% enzyme replacement level. Tubes were vortexed twice daily. Twelve replicates per strain were dosed for each time point.

The MeJi697 strain was constructed by inserting the expression constructs at the location of XII-5 in the yeast genome in the MBG4931 parent strain.

At 21, 44.5, and 60 hours, 48 tubes were sampled by weighing, adding 150 μl of 40% H₂SO₄, and centrifugation for 10 minutes at 3500 rpm. Supernatant was then filtered through a 0.2 μM filter prior to being diluted in mobile phase for HPLC analysis.

Glucoamylase blend A: Blend comprising Talaromyces emersonii glucoamylase disclosed as SEQ ID NO: 34 in WO99/28448, Trametes cingulata glucoamylase disclosed as SEQ ID NO: 2 in WO 06/69289, and Rhizomucor pusillus alpha-amylase with Aspergillus niger glucoamylase linker and starch binding domain (SBD) disclosed in SEQ ID NO: 5 herein having the following substitutions G128D+D143N using SEQ ID NO: 5 numbering (activity ratio in AGU:AGU:FAU-F is about 29:8:1).

### Results:

This experiment tested the performance of a GsAMG expressing strain with a 50% reduction in exogenous enzyme glucoamylase. MeJi697 dosed at 50% of the industrially recommended enzyme dose showed statistically identical performance to the parent strain at full dose at 60 hours of fermentation. The performance of this strain was also statistically identical to Transferm® Yield+ (commercial yeast strain expressing a glucoamylase available from Mascoma) at this time point. At 44.5 hours, MeJi697 showed higher ethanol titers than the parent strain at full dose and Transferm Yield+at comparable dose.

### Example 4: Application Performance of a Yeast Expressing a Glucoamylase (AMG) in an SSF Process (MBG4911 Expressing AMG)

Mash Preparation: Ground yellow dent corn was mixed with tap water and the dry solids (DS) level was determined to be 35.4% by moisture balance. This mixture was supplemented with 3 ppm LACTROL® and 500 ppm urea. The slurry was adjusted to pH 4.5 with 40% H₂SO₄.

Yeast Strains and Preparation: The two yeast strains tested in these experiments were MBG4911 and AgJg013 (MBG4911 expressing PsAMG). AgJg013 was constructed in a similar manner to that shown in Example 1 above. Yeasts were propagated in filter sterilized liquid media (2% w/v D-glucose, 1% peptone, and 0.5% yeast extract). Using a sterile loop under a UV hood, cells from a lawn were transferred into 60 mL of the liquid media in 125 mL sterile vented flask and incubated at 150 rpm in a 32° C. air shaker. Cells were harvested at 18 hours by spinning in 50 ml centrifuge tubes at 3000rpm for 10 minutes and decanting the supernatant. Cells were washed once in 25 ml of water and the resulting cell pellet was resuspended in 1.5 ml tap water. Total yeast concentration was determined using the YC-100 in duplicate.

Simultaneous Saccharification and Fermentation (SSF): Approximately 5 grams of mash was transferred to test tubes having a 1/64 hole drilled in the top to allow CO₂ release. PE096 was dosed to each tube of mash at 0.028 FauF/ gDS. PsAMG was dosed at 0.45 AGU/gDS, 0.23 AGU/gDS, 0.15 AGU/gDS, or omitted entirely. Yeast was dosed at 5e6 cells/g mash. Milli-Q water was added to each tube so that a total volume of liquid added (enzyme+MQ water) to each tube would be equally proportionate to the mash weight. Fermentations took place in a 32° C. incubator for 72 hours. Samples were vortexed periodically (in the morning and in the evening) throughout the fermentation. Six replicates were run per treatment.

HPLC analysis: Fermentation sampling took place after 72 hours of fermentation. Each tube was processed for HPLC analysis by deactivation with 150 μL of 40% v/v H₂SO₄, vortexing, centrifuging at 1460×g for 10 minutes, and filtering through a 0.2 μm SpinX column. All samples were processed at a 5× dilution. Samples were stored at 4° C. prior to and during HPLC analysis.

TABLE 6
HPLC System and conditions

| | |
|---|---|
| HPLC System | Agilent's 1100/1200 series with |
| | Chem station software |
| | Degasser, Quaternary Pump, Auto-Sampler, |
| | Column Compartment/w |
| | Heater |
| | Refractive Index Detector (RI) |
| Column | Waters Ion Exclusion Column |
| | 150 mm × 7.8 mm |
| | Part#: WAT010295 |
| | Waters Guard Cartridge SH-1011P 50 mm × 6 mm |
| | Part #: WAT034243 |
| Method | 0.0005M H2SO4 mobile phase |
| | Flow rate: 1 ml/min |
| | Column temperature: 75° C. |
| | RI detector temperature: 40° C. |

34

Samples were analyzed for sugars (DP4+, DP3, DP2, glucose, and fructose), organic acids (lactic and acetic), glycerol, and ethanol.

Increased ethanol titers at 72 hours for AgJg013 compared to parent strain MBG4911 are shown in Table 7 below.

TABLE 7
Ethanol Titers at 72 Hours

| | MBG4911 | AgJg013 |
|---|---|---|
| Condition | Ethanol Titer (w/v %) | Ethanol Titer (w/v %) |
| PsAMG 0.45 AGU/gDS | 14.07 | 14.36 |
| PsAMG 0.23 AGU/gDS | 13.51 | 14.05 |
| PsAMG 0.15 AGU/gDS | 12.99 | 13.94 |
| PsAMG 0.00 AGU/gDS | 8.28 | 13.43 |

### Example 5: Application Performance of a Yeast Expressing a Glucoamylase (AMG) in an SSF Process (MBG4911 Expressing AMG)

Mash Preparation: Ground yellow dent corn was mixed with tap water and the dry solids (DS) level was determined to be 37.4% by moisture balance. This mixture was supplemented with 3 ppm LACTROL® and 500 ppm urea. The slurry was adjusted to pH 4.5 with 40% H₂SO₄. Water was added to bring the final dry solids to 35.0%.

Yeast Strains and Preparation: The two yeast strains tested in these experiments, MBG4911 and AgJg013 (MBG4911 expressing PsAMG), were prepared as described in Example 4.

Simultaneous Saccharification and Fermentation (SSF): Approximately 5 grams of mash was transferred to test tubes having a 1/64 hole drilled in the top to allow CO₂ release. PE096 was dosed to each tube of mash at 0.028 FauF/ gDS. PsAMG was dosed at 0.45 AGU/gDS, or 0.23 AGU/gDS. Yeast was dosed at 5e6 cells/g mash. Milli-Q water was added to each tube so that a total volume of liquid added (enzyme+MQ water) to each tube would be equally proportionate to the mash weight. Fermentations took place in a 32° C. incubator for 72 hours. Samples were vortexed periodically (in the morning and in the evening) throughout the fermentation. Seven replicates were run per treatment.

HPLC analysis: Fermentation sampling took place after 48 and 72 hours of fermentation. Each tube was processed for HPLC analysis as described in Example 4.

Increased ethanol titers at 72 hours for AgJg013 compared to parent strain MBG4911 are shown in Table 8 below. 50% of exogenous GA is able to be removed using the PsAMG expressing yeast.

TABLE 8
Ethanol titers at 72 Hours

| Enzyme | Yeast | 72 Hours (w/v %) |
|---|---|---|
| 0.45 AGU/gDS PsAMG | MBG4911 | 16.27 |
| 0.45 AGU/gDS PsAMG | AgJg013 | 16.46 |
| 0.23 AGU/gDS PsAMG | MBG4911 | 15.54 |
| 0.23 AGU/gDS PsAMG | AgJg013 | 16.37 |

### Example 6: Application Performance of a Yeast Expressing an Alpha-Amylase (AA) in an SSF Process (MBG4911 Expressing AA)

Mash Preparation: Ground yellow dent corn was mixed with tap water and the dry solids (DS) level was determined to be 35.06% by moisture balance. This mixture was supplemented with 3 ppm LACTROL® and 500 ppm urea. The slurry was adjusted to pH 4.5 with 40% H₂SO₄.

Yeast Strains and Preparation: The two yeast strains tested in these experiments were MBG4911 and MLBA795 (MBG4911 expressing PE096) were prepared as described in Example 4.

Simultaneous Saccharification and Fermentation (SSF): Approximately 5 grams of mash was transferred to test tubes having a 1/64 hole drilled in the top to allow CO₂ release. PE096 was dosed to a control set of tubes of mash for MBG4911 at 0.028 FauF/ gDS and was omitted entirely from the tubes for MLBA795. PsAMG was dosed at 0.45 AGU/gDS, or 0.23 AGU/gDS. Yeast was dosed at 10e6 cells/g mash. Milli-Q water was added to each tube so that a total volume of liquid added (enzyme+MQ water) to each tube would be equally proportionate to the mash weight. Fermentations took place in a 32° C. incubator for 72 hours. Samples were vortexed periodically (in the morning and in the evening) throughout the fermentation. Ten replicates were run per treatment.

HPLC analysis: Fermentation sampling took place after 72 hours of fermentation. Each tube was processed for HPLC analysis as described in Example 4.

Increased ethanol titers at 72 hours for strain MLBA795 compared to parent strain MBG4911 are shown in Table 9 below. With all exogenous AA removed, at 72 hours of fermentation 50% of exogenous GA is able to be removed using these AA expressing yeasts. When full enzyme dose is used, a boost in ethanol titer is observed.

TABLE 9
Ethanol Titers at 72 Hours

| Exogenous GA | Exogenous AA | | 72 Hours |
|---|---|---|---|
| (PsAMG) | (PE096) | Yeast | (w/v %) |
| 0.45 AGU/gDS | 0.028 FauF/g DS | MBG4911 | 14.27 |
| | AAPE096 | | |
| 0.45 AGU/gDS | No Exogenous AA | MBG4911 | 6.20 |
| 0.45 AGU/gDS | No Exogenous AA | MLBA795 | 14.55 |
| 0.23 AGU/gDS | No Exogenous AA | MBG4911 | 5.33 |
| 0.23 AGU/gDS | No Exogenous AA | MLBA795 | 14.34 |

### Example 7: Application Performance of a Yeast Expressing an Alpha-Amylase (AA) and a Glucoamylase (AMG) in an SSF Process (MBG4911 Expressing AA+AMG)

Mash Preparation: Ground yellow dent corn was mixed with tap water and the dry solids (DS) level was determined to be 37.4% by moisture balance. This mixture was supplemented with 3 ppm LACTROL® and 500 ppm urea. The slurry was adjusted to pH 4.5 with 40% H₂SO₄. Water was added to bring the final solids to 35.0% DS

Yeast Strains and Preparation: The two yeast strains tested in these experiments were MBG4911 and MLBA821 (MBG4911 expressing PsAMG and PE096). Yeast were propagated in filter sterilized liquid media (2% w/v D-glucose, 1% peptone, and 0.5% yeast extract). Using a sterile loop under a UV hood, cells from a lawn were transferred into 60 mL of the liquid media in 125 mL sterile vented flask and incubated at 150 rpm in a 32° C. air shaker. Cells were harvested at 18 hours by spinning in 50 ml centrifuge tubes at 3000rpm for 10 minutes and decanting the supernatant. Cells were washed once in 25 ml of water and the resulting cell pellet was resuspended in 1.5 ml tap water. Total yeast concentration was determined using the YC-100 in duplicate.

Simultaneous Saccharification and Fermentation (SSF): Approximately 5 grams of mash was transferred to test tubes having a 1/64 hole drilled in the top to allow CO₂ release. PE096 was dosed to tubes of mash for MBG4911 at 0.028 FauF/ gDS and was omitted entirely from the tubes for MLBA821. PsAMG was dosed at 0.45 AGU/gDS, or 0.23 AGU/gDS for MBG4911 and 0.23 AGU/gDS, 0.15 AGU/gDS, or 0.045 AGU/gDS for MLBA821. Yeast was dosed at 10e6 cells/g mash. Milli-Q water was added to each tube so that a total volume of liquid added (enzyme +MQ water) to each tube would be equally proportionate to the mash weight. Fermentations took place in a 32° C. incubator for 72 hours. Samples were vortexed periodically (in the morning and in the evening) throughout the fermentation. Ten replicates were run per treatment. HPLC analysis: Fermentation sampling took place after 72 hours of fermentation. Each tube was processed for HPLC analysis as described in Example 4.

Increased ethanol titers at 72 hours for strain MLBA821 compared to parent strain MBG4911 are shown in Table 10 below. The strain expressing both GA and AA is statistically identical to the host strain at full enzyme dose when dosed at only 10% of the exogenous GA with no added AA.

TABLE 10
Ethanol Titers at 72 Hours of Fermentation

| | | | | Tukey-Kramer |
|---|---|---|---|---|
| | | | | Connecting |
| Exogenous | Exogenous | | 72 | Letter Report |
| GA | AA | | Hours | (95% confidence |
| (PsAMG) | (PE096) | Yeast | (w/v %) | level) |
| 0.45 AGU/gDS | 0.028 FauF/g DS | MBG4911 | 16.27 | A B |
| 0.23 AGU/gDS | 0.028 FauF/g DS | MBG4911 | 15.54 | C |
| 0.23 AGU/gDS | No Exogenous AA | MLBA821 | 16.43 | A |
| 0.15 AGU/gDS | No Exogenous AA | MLBA821 | 16.25 | A B |
| 0.045 AGU/gDS | No Exogenous AA | MLBA821 | 16.10 | B |

### Example 8: Application Performance of a Yeast Expressing an Alpha-Amylase (AA) and a Cilucoamylase (AMG) in an SSF Process (MBG4911 Expressing AA+AMG)

Mash Preparation: Ground yellow dent corn was mixed with tap water and the dry solids (DS) level was determined to be 35.3% by moisture balance. This mixture was supplemented with 3 ppm LACTROL® and 500 ppm urea. The slurry was adjusted to pH 4.5 with 40% H₂SO₄. Water was added to bring the final solids to 35.0% DS

Yeast Strains and Preparation: Yeast strains ER, MBG4911, AgJg013, MLBA855, PsinER1 and MLBA888 were tested in this experiment. Yeast were propagated in filter sterilized liquid media (6% w/v D-glucose, 1% peptone, and 0.5% yeast extract). Using a sterile loop under a UV hood, cells from a lawn were transferred into 60 mL of the liquid media in 125 mL sterile vented flask and incubated at 150 rpm in a 32° C. air shaker. Cells were harvested at 18 hours by spinning in 50 ml centrifuge tubes at 3000rpm for 10 minutes and decanting the supernatant. Cells were washed once in 25 ml of water and the resulting cell pellet was resuspended in 1.5 ml tap water. Total yeast concentration was determined using the YC-100 in duplicate.

Simultaneous Saccharification and Fermentation (SSF): Approximately 200 grams of mash was transferred to 250 ml Wheaton bottles with a 1/64 hole drilled in the top to allow CO₂ release. PE096 was dosed to bottles of mash for ER, MBG4911, AgJg013, and PsinER 1 at 0.025 FauF/ gDS and was omitted entirely from the bottles for MLBA855 and MLBA888. PsAMG was dosed at 0.4 AGU/gDS for ER and MBG4911 and 0.2 AGU/gDS, or 0.1 AGU/gDS for the remaining strains. Yeast was dosed at 5e6 cells/g mash. Milli-Q water was added to each tube so that a total volume of liquid added (enzyme+MQ water) to each tube would be equally proportionate to the mash weight. Fermentations took place in a 32° C. incubator for 91 hours. Samples were swirled periodically (in the morning and in the evening) throughout the fermentation. Three replicates were run per treatment.

HPLC analysis: Fermentation sampling took place after 72 and 91 hours of fermentation. Each tube was processed for HPLC analysis as described in Example 4.

Ethanol titers at 72 hours for strains AgJg013 and MLBA855 compared to parent strain MBG4911, and PsinER1 and MLBA888 compared to parent strain ER are shown in Table 11 below. The MBG4911 based strains expressing either GA or both GA and AA were able to ferment to statistically identical levels as the parent strain when dosed at 50% or 25% of the full GA dose. The combination GA and AA strains required no added AA to reach these levels. The ER based strains were able to ferment to statistically identical levels as the parent at 50% of the full GA dose. Again the combination strain required no added AA to do so. The combination strain was also able to ferment to statistically identical levels as the parent strain at 25% of the full GA dose.

TABLE 11
Ethanol Titers at 72 hours

| | | | | Tukey |
|---|---|---|---|---|
| | | | | Kramer |
| | | | | Connecting |
| Exogenous | Exogenous | | ETOH | Letter |
| GA | AA | | Titer | Report (95% |
| (PsAMG) | (PE096) | Yeast | (% w/v) | Confidence) |
| 0.2 AGU/g DS | No Exogenous AA | MLBA855 | 18.1 | A |
| 0.2 AGU/g DS | 0.025 FauF/gDS | AgJg013 | 18.1 | A |
| 0.1 AGU/g DS | No Exogenous AA | MLBA855 | 18.1 | A |
| 0.2 AGU/g DS | No Exogenous AA | MLBA888 | 18.1 | A |
| 0.4 AGU/gDS | 0.025 FauF/gDS | ER | 18.0 | A |
| 0.4 AGU/gDS | 0.025 FauF/gDS | MBG4911 | 18.0 | AB |
| 0.1 AGU/g DS | 0.025 FauF/gDS | AgJg013 | 17.9 | AB |
| 0.1 AGU/g DS | No Exogenous AA | MLBA888 | 17.9 | AB |
| 0.2 AGU/g DS | 0.025 FauF/gDS | PsinER1 | 17.8 | AB |
| 0.1 AGU/g DS | 0.025 FauF/gDS | PsinER1 | 17.6 | B |

Ethanol titers at 91 hours for strains AgJg013 and MLBA855 compared to parent strain MBG4911, and PsinER1 and MLBA888 compared to parent strain ER are shown in Table 12 below. The MBG4911 based strains expressing either GA or both GA and AA were able to ferment to statistically identical levels as the parent strain when dosed at 50% or 25% of the full GA dose. The combination GA and AA strains required no added AA to reach these levels. The ER based strains were able to ferment to statistically identical levels as the parent at 50% of the full GA dose. Again the combination strain required no added AA to do so. It is also noted that the MBG4911 strains all outperformed the ER based strains at this time point.

TABLE 12
Ethanol Titers at 91 hours

| | | | | Tukey |
|---|---|---|---|---|
| | | | | Kramer |
| | | | | Connecting |
| Exogenous | Exogenous | | ETOH | Letter |
| GA | AA | | Titer | Report (95% |
| (PsAMG) | (PE096) | Yeast | (% w/v) | Confidence) |
| 0.1 AGU/g DS | 0.025 FauF/gDS | AgJg013 | 19.4 | A |
| 0.1 AGU/g DS | No Exogenous AA | MLBA855 | 19.3 | A |
| 0.2 AGU/g DS | 0.025 FauF/gDS | AgJg013 | 19.3 | A |
| 0.4 AGU/gDS | 0.025 FauF/gDS | MBG4911 | 19.3 | A |
| 0.2 AGU/g DS | No Exogenous AA | MLBA855 | 19.2 | A |
| 0.4 AGU/gDS | 0.025 FauF/gDS | ER | 18.7 | B |
| 0.2 AGU/g DS | 0.025 FauF/gDS | PsinER1 | 18.6 | BC |
| 0.2 AGU/g DS | No Exogenous AA | MLBA888 | 18.4 | BC |
| 0.1 AGU/g DS | 0.025 FauF/gDS | PsinER1 | 18.3 | C |
| 0.1 AGU/g DS | No Exogenous AA | MLBA888 | 18.3 | C |

### Example 9: Application Performance of a Yeast Expressing an Alpha-Amylase (AA) and a Glucoamylase (AMG) in an SSF Process (MBG4931 Expressing AA+AMG)

A liquefied mash prepared using a commercial bacterial alpha-amylase product, Avantec Amp, was obtained from a commercial plant, Valero Albert City and used for this experiment. Mash solids were read to be 33.22% using a moisture balance. Approximately 5 grams of corn mash was fermented in pre- weighed 15 ml flip top conical tubes with a small hole drilled for gas release. A saccharification composition, Glucoamylase blend A dosed at 0.6 AGU/gDS for the parent strain (MBG4931) as the full enzyme dose. A strain expressing the GsAMG (MeJi705; constructed in a similar manner to that shown in Example 1 above) and two strains expressing GsAMG and PE096 (MLBA889, comprising 6X GsAMG, 2X PE096; and MLBA891, comprising 4×GsAMG, 4×Pe096; both prepared in manners similar to those described herein) were all dosed at 0.3 AGU/gDS to test a 50% enzyme replacement level or 0.15 AGU/gDS to test a 75% enzyme replacement level. Tubes were vortexed twice daily. Five replicates per strain were dosed for each condition.

At 52 hours, tubes were sampled by weighing, adding 150 μl of 40% H₂SO₄, and centrifugation for 10 minutes at 3500 rpm. Supernatant was then filtered through a 0.2 μM filter prior to being diluted in mobile phase for HPLC analysis.

This experiment tested the performance of a GsAMG and GsAMG/Pe096 expressing strains with a 50% reduction in exogenous enzyme glucoamylase. MeJi705, MLBA889, and MLBA891 dosed at 50% of the industrially recommended enzyme dose showed statistically identical performance to the parent strain at full dose at 52 hours of fermentation. The strains expressing both GsAMG and Pe096 (MLBA889 and MLBA891) dosed at 25% of the industrially recommended enzyme dose also showed statistically identical performance to the parent strain at full dose at this time point, indicating a benefit from the addition of the Pe096 expression over the GsAMG expression alone.

### Example 10: Application Performance of a Yeast Expressing a Glucoamylase (AMG) in an SSF Process (MBG4931 Expressing AA)

A liquefied mash prepared using a commercial bacterial alpha-amylase product, Liquozyme LpH, was obtained from a commercial plant, Little Sioux and used for this experiment. Mash solids were read to be 34.5% using a moisture balance. Approximately 5 grams of corn mash was fermented in pre- weighed 15 ml flip top conical tubes with a small hole drilled for gas release. A saccharification composition, Glucoamylase blend A dosed at 0.6 AGU/gDS for the control strain (ER) as the full enzyme dose. A strain expressing GtAMG (MHCT408; prepared in a manner similar to that described above) and parent strain MBG4931 were all dosed at 0.6, 0.45, 0.35, 0.25 and 0.15 AGU/gDS to test different enzyme replacement levels. Tubes were vortexed twice daily. Four replicates per strain were dosed for each condition.

At 54 hours, tubes were sampled by weighing, adding 50 pl of 40% H₂SO₄, and centrifuged for 10 minutes at 3500 rpm. Supernatant was then filtered through a 0.2 μM filter prior to being diluted in mobile phase for HPLC analysis.

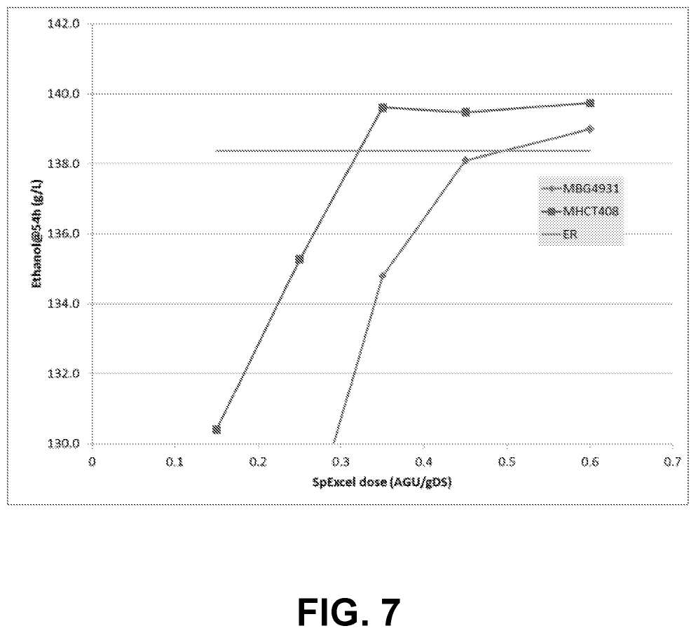

This experiment tested the performance of a GtAMG with different reductions in exogenous enzyme glucoamylase. Results are shown in Table 13 and FIG. 7. MHCT408 dosed at 42% of the industrially recommended enzyme dose showed statistically higher performance to the control strain at full dose at 54 hours of fermentation.

TABLE 13
Ethanol Titers at 54 hours

| Dose | Ethanol Titers (g/L) | | |
|---|---|---|---|
| (AGU/gDS) | MBG4931 | MHCT408 | ER |
| 0.6 | 139.0 | 139.7 | 138.4 |
| 0.45 | 138.1 | 139.5 | |
| 0.35 | 134.8 | 139.6 | |
| 0.25 | 126.7 | 135.3 | |
| 0.15 | 117.9 | 130.4 | |

1-20 (canceled)

21. A process of producing ethanol from starch-containing material comprising:
(a) saccharifying the starch-containing material; and
(b) fermenting using a fermentation organism;
wherein the fermenting organism is a Saacaromyces cerevisiae strain selected from:
MBG4931 (deposited under Accession No. V15/004036 at National Measurement Institute, Victoria, Australia) further comprising an expression construct encoding a glucoamylase and/or an alpha-amylase;
MBG4851 (deposited under Accession No. V14/004037 at National Measurement Institute, Victoria, Australia) further comprising an expression construct encoding a glucoamylase and/or an alpha-amylase;
MBG4911 (deposited as V15/001459 at National Measurement Institute, Victoria, Australia) further comprising an expression construct encoding a glucoamylase and/or an alpha-amylase;
MBG4913 (deposited as V15/001460 at National Measurement Institute, Victoria, Australia) further comprising an expression construct encoding a glucoamylase and/or an alpha-amylase; or
MBG4914 (deposited as V15/001461 at National Measurement Institute, Victoria, Australia) further comprising an expression construct encoding a glucoamylase and/or an alpha-amylase.

22. The process of claim 21, wherein the fermenting organism comprises an expression construct encoding a glucoamylase.

23. The process of claim 22, wherein the glucoamylase is a Gloeophyllum glucoamylase, a Trametes glucoamylase or a Pycnoporus glucoamylase.

24. The process of claim 22, wherein the glucoamylase comprises an amino acid sequence having at least 80% sequence identity to the polypeptide of SEQ ID NO: 1, 2, 3, or 4.

25. The process of claim 22, wherein the glucoamylase comprises or consists of the amino acid sequence of SEQ ID NO: 1, 2, 3, or 4.

26. The process of claim 21, wherein the fermenting organism comprises an expression construct encoding an alpha-amylase.

27. The process of claim 26, wherein the alpha-amylase is a Rhizomucor pusillus alpha-amylase or an Aspergillus terreus alpha-amylase.

28. The process of claim 26, wherein the alpha-amylase is a Rhizomucor pusillus alpha-amylase with an Aspergillus niger glucoamylase linker and starch-binding domain (SBD).

29. The process of claim 26, wherein the alpha-amylase is a Rhizomucor pusillus alpha-amylase with an Aspergillus niger glucoamylase linker and starch-binding domain (SBD).

30. The process of claim 26, wherein the alpha-amylase comprises an amino acid sequence having at least 80% sequence identity to the polypeptide of SEQ ID NO: 5 or 6.

31. The process of claim 26, wherein the alpha-amylase comprises or consists of the amino acid sequence of SEQ ID NO: 5 or 6.

32. The process of claim 21, wherein step (a) and step (b) are carried out as a simultaneous saccharification and fermentation process (SSF).

33. The process of claim 21, wherein the saccharification step (a) is conducted above the initial gelatinization temperature.

34. The process of claim 21, wherein the saccharification step (a) is conducted below the initial gelatinization temperature.

35. A Saacaromyces cerevisiae strain selected from:
MBG4931 (deposited under Accession No. V15/004036 at National Measurement Institute, Victoria, Australia) further comprising an expression construct encoding a glucoamylase and/or an alpha-amylase;
MBG4851 (deposited under Accession No. V14/004037 at National Measurement Institute, Victoria, Australia) further comprising an expression construct encoding a glucoamylase and/or an alpha-amylase;
MBG4911 (deposited as V15/001459 at National Measurement Institute, Victoria, Australia) further comprising an expression construct encoding a glucoamylase and/or an alpha-amylase;
MBG4913 (deposited as V15/001460 at National Measurement Institute, Victoria, Australia) further comprising an expression construct encoding a glucoamylase and/or an alpha-amylase; and
MBG4914 (deposited as V15/001461 at National Measurement Institute, Victoria, Australia) further comprising an expression construct encoding a glucoamylase and/or an alpha-amylase.

36. The yeast strain of claim 35, wherein the fermenting organism comprises an expression construct encoding a glucoamylase.

37. The yeast strain of claim 36, wherein the glucoamylase is a Gloeophyllum glucoamylase, a Trametes glucoamylase or a Pycnoporus glucoamylase.

38. The yeast strain of claim 36, wherein the glucoamylase comprises an amino acid sequence having at least 80% sequence identity to the polypeptide of SEQ ID NO: 1, 2, 3, or 4.

39. The yeast strain of claim 36, wherein the glucoamylase comprises or consists of the amino acid sequence of SEQ ID NO: 1, 2, 3, or 4.

40. The yeast strain of claim 35, wherein the fermenting organism comprises an expression construct encoding an alpha-amylase.

41. The yeast strain of claim 40, wherein the alpha-amylase is a Rhizomucor pusillus alpha-amylase or an Aspergillus terreus alpha-amylase.

42. The yeast strain of claim 40, wherein the alpha-amylase is a Rhizomucor pusillus alpha-amylase with an Aspergillus niger glucoamylase linker and starch-binding domain (SBD).

43. The yeast strain of claim 40, wherein the alpha-amylase is a Rhizomucor pusillus alpha-amylase with an Aspergillus niger glucoamylase linker and starch-binding domain (SBD).

44. The yeast strain of claim 40, wherein the alpha-amylase comprises an amino acid sequence having at least 80% sequence identity to the polypeptide of SEQ ID NO: 5 or 6.

45. The yeast strain of claim 40, wherein the alpha-amylase comprises or consists of the amino acid sequence of SEQ ID NO: 5 or 6.
